(19) United States
(12) Patent Application Publication
(10) Pub. No.: US 2012/0021028 A1
(43) Pub. Date: Jan. 26, 2012

(54) Powdery Composition for Shaping Keratin Fibers and Giving Them Shine

(76) Inventors: Thorsten Knappe, Schenefeld (DE); Bernd Richters, Hamburg (DE); Rolf Bayersdoerfer, Hamburg (DE); Dirk Hentrich, Hamburg (DE)

(21) Appl. No.: 13/253,304

(22) Filed: Oct. 5, 2011

Related U.S. Application Data
(63) Continuation of application No. PCT/EP2010/053700, filed on Mar. 22, 2010.

(30) Foreign Application Priority Data
Apr. 7, 2009 (DE) 102009002267.8

(51) Int. Cl. A61K 8/02; A61Q 5/06

(52) U.S. Cl. 424/401

(57) ABSTRACT

Powdery compositions for temporarily shaping human hair containing 50 to 90 wt. % of water, 0.5 wt. % to 10 wt. % of at least one other liquid having a boiling point higher than 150° C. at a pressure of 1013 mbar, at least one starch substituted with carboxy-(C₁ to C₄) alkyl groups, and metal oxide particles coated on the surface with fluorine-containing organic groups. The powdery compositions, when applied to hair, provide excellent hairstyle and shine. The powdery compositions can easily be applied to hair. By mechanically manipulating hair covered with the powdery compositions, cosmetic active substances are released in a targeted manner on the areas of the hair to be shaped

### CROSS REFERENCE TO RELATED APPLICATIONS

This is a continuation of International Patent Application No. PCT/EP2010/053700 filed 22 Mar. 2010, which claims priority to German Patent Application No. 10 2009 002 267.8 filed 7 Apr. 2009, both of which are incorporated herein by reference.

The present invention relates to powdery compositions for temporarily shaping human hair, containing 50 to 90 wt. % of water, 0.5 wt. % to 10 wt. % of at least one further liquid having a boiling point higher than 150° C. at a pressure of 1013 mbar, at least one starch substituted with carboxy-(C₁ to C₄) alkyl groups, and metal oxide particles, which have been coated on the surface with fluorine-containing organic groups. A method for temporary shaping with this composition and the use of this composition for giving shine to and for temporarily shaping human hair are also included.

Styling agents for shaping keratinic fibers have long been known and used in various embodiments to construct, refresh and set hairstyles, which for many hair types can only be achieved by use of active fixing agents. Hair treatment agents used to permanently shape the hair as well as those used for its temporary shaping play an important role here. Temporary shaping effects which offer good hold without adversely affecting the healthy appearance of the hair (e.g., its shine) can be achieved, for example, by hair sprays, hair waxes, hair gels, blow-drying, etc.

In many cases known forms of temporary styling agents cannot be metered out with satisfactory accuracy. Thus, hair gels, hair creams and hair waxes, for instance, are difficult to distribute once they have been applied to the hair. As soon as a comb or hands to which the styling agent has been applied come into contact with the first sections of hair, comparatively large amounts of styling agent are released onto the hair. By contrast, comparatively little styling agent is worked into sections of hair which are only reached later by the comb or the hands. As a consequence, the user must initially apply a large amount of styling agent so that those sections of hair reached last also receive sufficient styling agent, or the user is forced to apply the styling agent in several stages, treating different sections of hair each time. Hair sprays can be distributed more evenly over the hair. However, as the user has no means of visually assessing the total amount of styling agent applied, there is a risk that more styling agent is applied to the hair than would actually be necessary.

In addition, known forms of temporary styling agents generally require a large amount of auxiliary substances for not only shaping the hairstyle but also formulating the agent. Thus, styling agents often contain large amounts of organic solvents. Hair spray formulations additionally requires further organic compounds which are used as blowing agents. This results in the environment being loaded with volatile organic compounds (VOC), while increasing considerably product volume and hence the volume of the necessary packaging.

The present invention therefore provides a hair treatment agent for temporarily shaping human hair which gives excellent and lasting styling result, is in a compact form, and can be metered out accurately and easily.

It was found that this can be achieved in a simple manner by a powdered styling agent.

Powdery cosmetics are known and have been used in the area of skin treatment, for instance, for quite some time. Typical examples include foundation powder or eye shadow. Use of a carrier material in powder form is necessary in order to achieve the powdery consistency. Silicon dioxide, for instance, can be used as a suitable carrier material. Hydrophobed silicon dioxide is of particular interest. This can be obtained, for example, from pyrogenic silicon dioxide, which is commercially available in various specifications. Untreated pyrogenic silicon dioxide bears silanol and siloxane groups on the surface. Therefore, it has a high affinity for water (i.e., it is hydrophilic). Alkylsilyl groups can bond chemically to the surface of the pyrogenic silicon dioxide by reaction with suitable organic silicon compounds. This results in modified silicon dioxide powders which are no longer wetted by water (i.e., they have hydrophobic properties).

Use of hydrophobic silicon dioxide in cosmetics to produce dry water for skin is described in Seifen, Öle, Fette, Wachse (SÖFW), 3 (2004), pp. 4-13. The hydrophobic properties of the modified silicon dioxide are utilized here, wherein the silicon dioxide does not simply disperse when intensively mixed with water. The water droplets are instead sheathed by the hydrophobic solid particles and prevented from coalescing again. In this way powdery solids with water content of up to over 95% can be obtained. Under mechanical loading (e.g., when rubbed on the skin), the entrapped water is released again. This dry water is described as the basis for producing stable, solid hydrogen peroxide and spreadable preparations having very low oil content.

This concept also underlies the production of cosmetic or pharmaceutical, liquefiable powder compositions described in EP 1 235 554 B1. The powder compositions contain hydrophobically coated metal oxide particles in which water and a water-soluble polymer are entrapped, with the compositions having less than 1% oil. The aim of adding the water-soluble polymer is to make the powder feel pleasant and non-grainy when applied to the skin, without the need to add an oil component to the product for that purpose. The polymer is added to the water phase in an amount from 0.01 to 5 wt. %, with a content of 0.1 to 1 wt. % being preferred. The liquefiable powder compositions can be used to produce decorative cosmetics. In addition, use in deodorants or sunscreen agents or application on the hair as the basis of hair treatment agents containing pearlescent agents or care components is also described. Their use in the area of styling agents is not mentioned.

Use of powdery compositions for temporary hair shaping is described in publication WO 2007/051511 A1. These powdery compositions contain hydrophobed silicon dioxide, 50 to 90 wt. % of water, and at least one film-forming and/or fixing polymer.

A disadvantage of known powdery compositions is that after application, the hair appears almost dull (i.e., it has no shine). The present invention, in addition to the fulfillment of the aforementioned parameters, additionally provides a shine-imparting composition for temporarily shaping hair.

The present invention therefore firstly provides powdery compositions for temporarily shaping human hair containing:
i) 50 to 90 wt. % of water,
ii) 0.5 wt. % to 10 wt. % of at least one further liquid, which has a boiling point higher than 150° C. at a pressure of 1013 mbar,
iii) at least one starch substituted with carboxy-(C₁ to C₄) alkyl groups, and
iv) metal oxide particles which have been coated on the surface with fluorine-containing organic groups.

Temporary shaping of human hair is understood to be the temporary fixing of a hairstyle. Hair is understood in this context by the person skilled in the art to be hair which grows on the head and is not rooted in the face. For example, eyebrows, beards and eyelashes are not encompassed by the term hair according to the present invention.

Within the meaning of the invention particles are particles of solids in granular form (cf. DIN 66160: 1992-09).

Compositions whose particles are free-flowing under their own weight are understood to be powdery within the meaning of the invention (cf. DIN EN ISO 6186: 1998-08).

Powdery compositions according to the invention can be metered out very easily. They can moreover be distributed very evenly in the hair, as the constituents of the powder and optionally active ingredients contained within it, which are liquid under application conditions, are released only under mechanical loading. The powder can therefore be distributed carefully in the hair first and only then subjected to stronger mechanical loading, for example by systematically massaging the powder into the hair. In this way the liquid phase is released only directly onto the desired section of hair. An excellent metering and action can be achieved very systematically in this way.

Powdery composition according to the invention preferably include core-shell particles whose shell contains metal oxide particles coated on the surface with fluorine-containing organic groups, and whose core contains a liquid, aqueous phase, wherein the powdery composition contains:
i) 50 to 90 wt. % of water,
ii) 0.5 wt. % to 10 wt. % of at least one further liquid having a boiling point higher than 150° C. at a pressure of 1013 mbar, and
iii) at least one starch substituted with carboxy-(C₁ to C₄) alkyl groups.

According to this preferred embodiment the core-shell particles of the powdery composition thus encompass a core having an aqueous, liquid phase. The core is thus in liquid form. This core is surrounded by a shell, which is based on separable individual particles of metal oxide coated on the surface with fluorine-containing organic groups.

Powdery compositions according to the invention contain as a mandatory component 50 to 90 wt. %, particularly 60 to 85 wt. % of water, based on total weight of the powdery composition.

Powdery compositions according to the invention further contain as a mandatory component 0.5 wt. % to 10 wt. % of at least one further liquid having a boiling point higher than 150° C. at a pressure of 1013 mbar.

In a preferred embodiment, liquids having a boiling point higher than 150° C. at a pressure of 1013 mbar that are used are dissolved in water at 20° C.

It is particularly preferable for this liquid having a boiling point higher than 150° C. at a pressure of 1013 mbar to be chosen from at least one (C₃ to C₈) alcohol having at least two hydroxyl groups.

This liquid having a boiling point higher than 150° C. at a pressure of 1013 mbar is most particularly preferably chosen from at least one compound from the group of glycerol, 1,3-butylene glycol, 1,4-butylene glycol, 1,2-pentanediol, 2-methyl-2,4-dihydroxypentane and 2-ethyl-1,3-hexanediol. Most preferably, the powdery composition contains glycerol as the liquid having a boiling point higher than 150° C. at a pressure of 1013 mbar.

As a further mandatory component the powdery composition according to the invention contains at least one starch substituted with carboxy-(C₁ to C₄) alkyl groups. According to the invention the starch substituted with carboxy-(C₁ to C₄) alkyl groups can likewise be used in the neutralized form of its salts, for example, as a sodium or potassium salt.

The starch substituted with carboxy-(C₁ to C₄) alkyl groups is preferably present in powdery compositions according to the invention in an amount of from 0.1 wt. % to 5 wt. %, particularly 0.75 wt. % to 3.5 wt. %, based on total weight of the powdery composition.

Starch is a reserve carbohydrate which is stored by many plants in the form of starch granules of conventionally 1 to 200 μm in size in various parts of the plant, for example, in tubers or roots, cereal seeds, fruits and in the pith. Starch that can be used according to the invention for producing the starch substituted with carboxy-(C₁ to C₄) alkyl groups can be obtained, for example, from potatoes, maize, rice, peas, acorns, chestnuts, barley, wheat, bananas, sago, millet, sorghum, oats, barley, rye, beans, yams, arrowroot or cassava.

Starch belongs to the homoglycan family and is a polycondensation product of D-glucose. Starch consists of three structurally different polymers—d-glucopyranose, namely amylose, amylopectin and an intermediate fraction. Relatively tall plants contain 0 to 45 wt. % of amylose relative to dry matter.

In a preferred embodiment the powdery composition contains as the starch substituted with carboxy-(C₁ to C₄) alkyl groups at least one compound chosen from the group of carboxymethyl starch, carboxyethyl starch, carboxypropyl starch. Carboxymethyl starch is most particularly preferred for use in powdery compositions according to the invention. Carboxymethyl starch is used, for example, in the form of the commercial product Covagel (carboxymethyl starch, sodium salt, with potato starch as the starch source; INCI name: Sodium Carboxymethyl Starch) from Sensient/LCW.

Powdery compositions according to the invention also contain metal oxide particles which have been coated on the surface with fluorine-containing organic groups. By definition, one skilled in the art understands fluorine-containing organic groups to be chemical molecules/molecule fragments comprising at least one carbon atom and at least one fluorine atom covalently bonded thereto. Within the meaning of the invention these fluorinated organic groups can be covalently bonded to the surface of the metal oxide particle or adsorbed at the surface of the metal oxide particle (e.g. in the form of an oil).

The powdery composition according to the invention preferably contains metal oxide particles coated on the surface with fluorine-containing organic groups in an amount of from 5 wt. % to 25 wt. %, based on total weight of the powdery composition.

It has further proved advantageous for metal oxide particles coated on the surface with fluorine-containing organic groups to have a particle diameter of 5 μm or less, preferably 1 μm or less, more preferably from 20 to 100 nm.

Metal oxide particles coated on the surface with fluorine-containing organic groups are preferably chosen from aluminates coated with fluorine-containing organic groups, silicates coated with fluorine-containing organic groups, aluminum silicates coated with fluorine-containing organic groups, titanium dioxide coated with fluorine-containing organic groups, and silica gel coated with fluorine-containing organic groups.

Particularly preferred aluminates coated with fluorine-containing organic groups are chosen from active aluminum oxide, alpha-aluminum oxide, beta-aluminum oxide, gamma-aluminum oxide and mixtures thereof, all cited aluminates being correspondingly coated with fluorine-containing organic groups.

Particularly preferred aluminum silicates (also known as alumosilicates) coated with fluorine-containing organic groups are chosen from phyllosilicates, tectosilicates, all cited aluminum silicates being correspondingly coated with fluorine-containing organic groups.

Preferably suitable phyllosilicates coated with fluorine-containing organic groups are selected from kaolins (particularly from kaolinite, dickite, hallosite and nacrite), serpentine, talc, pyrophyllite, montmorillonite, quartz, bentonite, mica (particularly from illite, muscovite, paragonite, phlogopite, biotite, lepidolite, margarite, smectite (particularly from montmorillonite, saponite, nontronite, hectorite)), all cited phyllosilicates being correspondingly coated with fluorine-containing organic groups.

Preferably suitable tectosilicates coated with fluorine-containing organic groups are chosen from feldspar minerals (particularly albite, orthoclase, anorthite, leucite, sodalite, hauyne, labradorite, lazurite, nosean, nepheline), all cited tectosilicates being correspondingly coated with fluorine-containing organic groups.

It is in turn particularly preferable for the metal oxide particles to be chosen from at least one representative of phyllosilicate coated on the surface with fluorine-containing organic groups (particularly talc coated on the surface with fluorine-containing organic groups, mica coated on the surface with fluorine-containing organic groups), titanium dioxide coated on the surface with fluorine-containing organic groups, and/or silicon dioxide coated on the surface with fluorine-containing organic groups.

Particularly suitable metal oxide particles coated on the surface with fluorine-containing organic groups are those which have been coated on the surface with perfluorinated organic compounds, particularly with perfluoroalkyl residues. Such metal oxides are preferred which have been hydrophobed with at least one residue chosen from perfluoroalkylsilyl, polyperfluoro-(C₂ to C₆) alkylene oxide, polysiloxane having perfluoroalkyl groups, perfluoroalkyl phosphate, polyfluoroalkyl phosphate ether. Of those, metal oxide particles coated on the surface with fluorine-containing organic groups are preferred which have polyperfluoro-(C₂ to C₆) alkylene oxide residues on the surface. These are preferably chosen from mica coated with polyperfluoro-(C₂ to C₆) alkylene oxide and/or titanium dioxide coated with polyperfluoro-(C₂ to C₆) alkylene oxide and/or silicon dioxide coated with polyperfluoro-(C₂ to C₆) alkylene oxide and/or talc coated with polyperfluoro-(C₂ to C₆) alkylene oxide.

The commercial products PW Covafluor® (LCW), PFS Talc JA 46-R® (Daito), Talc JA R46PF® (LCW), Submica M® (LCW) can particularly preferably be used.

Powdery compositions according to the invention most particularly preferably contain such metal oxide particles coated on the surface with fluorine-containing organic groups which have been coated on the surface by reaction with at least one reagent of formula (I)—

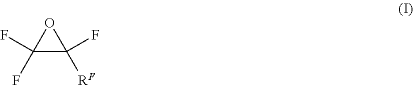

wherein R^{F} is a perfluoro-(C₁ to C₄) alkyl group, particularly trifluoromethyl.

Metal oxide particles which have been coated with reagents of formula (I) contain polyperfluoro-(C₂ to C₆) alkylene oxide residues covalently bonded to the surface.

It is furthermore preferable according to the invention for the powdery composition to contain such metal oxide particles coated on the surface with fluorine-containing organic groups which are coated on the surface with at least one compound of formula (Ia)—

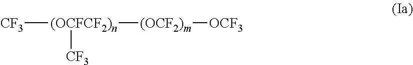

wherein the sum of n+m is a whole number from 20 to 80. These compounds have the INCI name Polyperfluoromethylisopropyl Ether. Compounds of formula (Ia) having a molecular weight from 1500 to 4000 g/mol are particularly preferably suitable.

The commercial products PW F-MS (nanofine titanium dioxide coated with polyperfluoromethylisopropyl ether (CAS no.: 69991-67-9, EINECS 274-225-4) and triethoxycaprylylsilane (INCI name: Cl 77891, Polyperfluoromethylisopropyl Ether, Triethoxycaprylylsilane) from Sensient/LCW can most particularly preferably be used.

It has proved preferable according to the invention to add to the powdery compositions, in addition to the metal oxide particles coated on the surface with fluorine-containing organic groups, silanized metal oxide particles bearing no fluorinated organic groups.

Powdery compositions of this embodiment preferably contain core-shell particles whose shell contains:
a) metal oxide particles coated on the surface with fluorine-containing organic groups (particularly those of the aforementioned preferred embodiments) and
b) silanized metal oxide particles (particularly those of the preferred embodiments mentioned below) and whose core contains a liquid, aqueous phase, wherein the powdery composition contains
i) 50 to 90 wt. % of water,
ii) 0.5 wt. % to 10 wt. % of at least one further liquid having a boiling point higher than 150° C. at a pressure of 1013 mbar, and
iii) at least one starch substituted with carboxy-(C₁ to C₄) alkyl groups.

According to this preferred embodiment core-shell particles of the powdery composition include a core having an aqueous, liquid phase. The core is thus liquid in form. This core is surrounded by a shell based on separable individual particles of metal oxide coated on the surface with fluorine-containing organic groups and of silanized metal oxide.

Silanized metal oxide particles are preferably chosen from silanized aluminates, silanized silicates, silanized aluminum silicates, silanized titanium dioxide and silanized silica gel.

Particularly preferred silanized aluminates are chosen from active silanized aluminum oxide, silanized alpha-aluminum oxide, silanized beta-aluminum oxide, silanized gamma-aluminum oxide and mixtures thereof.

Particularly preferred silanized aluminum silicates (also known as alumosilicates) are chosen from silanized phyllosilicates, silanized tectosilicates.

Preferred suitable silanized phyllosilicates are chosen from silanized kaolins (in particular, from silanized kaolinite, silanized dickite, silanized hallosite and silanized nacrite), silanized serpentine, silanized talc, silanized pyrophyllite, silanized montmorillonite, silanized quartz, silanized bentonite, silanized mica (in particular, from silanized illite, silanized muscovite, silanized paragonite, silanized phlogopite, silanized biotite, silanized lepidolite, silanized margarite, silanized smectite (particularly from silanized montmorillonite, silanized saponite, silanized nontronite, silanized hectorite)).

Preferred suitable silanized tectosilicates are chosen from silanized feldspar minerals (particularly silanized albite, silanized orthoclase, silanized anorthite, silanized leucite, silanized sodalite, silanized hauyne, silanized labradorite, silanized lazurite, silanized nosean, and silanized nepheline).

The additional silanized metal oxide particles preferably have a particle diameter of 5 μm or less, preferably 1 μm or less, particularly preferably from 20 to 100 nm.

It is particularly preferable for silanized metal oxide particles additionally present in the powdery composition according to the invention to be chosen from metal oxide particles which have been coated by reaction with at least one reagent of formula (II)—

(R¹O)ₙ(CH₃)₃₋ₙSi—R² (II)

wherein
R¹ is methyl or ethyl,
R² is a (C₁ to C₁₄) alkyl group, particularly a (C₆ to C₁₂) alkyl group, and
n is 1, 2 or 3.

Such reagents of formula (II) are preferred wherein n is the number 3.

In particular, it is particularly preferable according to the invention for the additional metal oxide particles to be chosen from at least one representative of phyllosilicate coated on the surface by reaction with at least one reagent of formula (II) (particularly talc coated on the surface by reaction with at least one reagent of formula (II), mica coated on the surface by reaction with at least one reagent of formula (II)), titanium dioxide coated on the surface by reaction with at least one reagent of formula (II), silicon dioxide coated on the surface by reaction with at least one reagent of formula (II).

It has been found that those coated by reaction with triethoxycaprylylsilane are particularly well suited as additional metal oxide particles. It is in turn particularly preferable for the metal oxide particles to be chosen from at least one representative of the group formed from phyllosilicate coated on the surface by reaction with triethoxycaprylylsilane (particularly talc coated on the surface by reaction with triethoxycaprylylsilane, mica coated on the surface by reaction with triethoxycaprylylsilane), titanium dioxide coated on the surface by reaction with triethoxycaprylylsilane, silicon dioxide coated on the surface by reaction with triethoxycaprylylsilane. Such metal oxide particles are available, for example, from Sensient/LCW under the trade names Mica 8 AS R0433 (mica coated by reaction with triethoxycaprylylsilane consisting of 99% mica, 1% triethoxycaprylylsilane; INCI name: Mica, Triethoxycaprylylsilane), Covapearl Bright 933 AS (pearlescent mineral pigments coated with triethoxycaprylylsilane and consisting of 25-35% titanium dioxide, 65-75% mica, 2% triethoxycaprylylsilane; INCI name: Cl 77891, Mica, Triethoxycaprylylsilane) (Sensient/LCW), PW Covasil S (INCI name: Titanium Dioxide, Trimethoxycaprylylsilane, Polymethyl methacrylate), Talc AS R0435 (talc coated by reaction with triethoxycaprylylsilane consisting of 99% talc and 1% triethoxycaprylylsilane; INCI name: Talc, Triethoxycaprylylsilane).

The powdery composition preferably contains metal oxide particles which have been coated on the surface by reaction with at least one reagent of formula (I) above and at least one reagent of formula (II) above. Here, in contrast to the previously described simple addition of silanized metal oxide particles to the metal oxide particles coated with fluorinated organic groups, the coatings coexist on the metal oxide particle.

Where powdery compositions according to the invention additionally contain the silanized metal oxide particles, it is preferable for the additional silanized metal oxide particles and the metal oxide particles coated on the surface with fluorine-containing organic groups to be present in a portion from 5 wt. % to 25 wt. % (relative to the weight of the powdery composition).

The powdery composition according to the invention can additionally contain at least one film-forming and/or fixing polymer to increase the hold of the hairstyle.

Film-forming polymers are understood to be polymers which leave behind a continuous film on the skin, hair or nails when they dry. Such film formers can be used in a wide range of cosmetic products, such as face masks, make-up, hair fixing agents, hair sprays, hair gels, hair waxes, hair tonics, shampoos or nail varnishes. Such polymers are preferred in particular which have an adequate solubility in water or water/alcohol mixtures in order for them to be present in the agent according to the invention in completely dissolved form. The film-forming polymers can be of synthetic or natural origin.

Film-forming polymers are also understood to be polymers which, when used in a 0.01 to 20 wt. % aqueous, alcoholic or aqueous-alcoholic solution, are capable of depositing a transparent polymer film on the hair.

Fixing polymers contribute to maintaining and/or establishing volume and fullness in the hairstyle as a whole. These polymers are simultaneously also film-forming polymers and are therefore generally typical substances for hair treatment agents used for shaping, such as hair fixing agents, hair mousses, hair waxes, and hair sprays. Film formation may be entirely localized here and may bond only a few fibers together.

The curl retention test or three-point bending test is frequently used as a test method for the fixing effect of a polymer.

Powdery compositions according to the invention preferably contain as film-forming and/or fixing polymer—

at least one cationic film-forming and/or cationic fixing polymer, and/or

at least one non-ionic film-forming and/or non-ionic fixing polymer, and/or

at least one anionic film-forming and/or anionic fixing polymer, and/or

at least one amphoteric film-forming and/or amphoteric fixing polymer.

In a further embodiment, the powdery compositions contain as film-forming and/or fixing polymer at least one cationic film-forming and/or cationic fixing polymer.

Cationic film-forming and/or cationic fixing polymers have at least one structural unit containing at least one permanently cationized nitrogen atom. Permanently cationized nitrogen atoms are understood to be nitrogen atoms bearing a positive charge and thus forming a quaternary ammonium compound. Quaternary ammonium compounds are mostly produced by reacting tertiary amines with alkylating agents, such as for example methyl chloride, benzyl chloride, dimethyl sulfate, dodecyl bromide, but also ethylene oxide. Depending on the tertiary amine used, the following groups are known in particular: alkyl ammonium compounds, alkenyl ammonium compounds, imidazolinium compounds and pyridinium compounds.

Preferred powdery compositions within the meaning of the invention contain film-forming cationic and/or fixing cationic polymers in an amount of from 0.1 wt. % to 8.0 wt. % particularly preferably from 0.2 wt. % to 7.0 wt. %, most particularly preferably from 0.5 wt. % to 5.0 wt. %, based on total weight of the composition.

Cationic film-forming and/or cationic fixing polymers can be chosen from cationic quaternized cellulose derivatives.

As a general rule, cationic quaternized celluloses bearing more than one permanent cationic charge in a side chain have proven to be advantageous within the meaning of the invention.

Of these, cationic cellulose derivatives should be mentioned which are produced by reacting hydroxyethyl cellulose with a dimethyl diallyl ammonium reactant (in particular dimethyl diallyl ammonium chloride), optionally in the presence of further reactants. Of these cationic celluloses, cationic celluloses with the INCI name Polyquaternium-4 are in turn particularly suitable, which are sold, for example, by National Starch under the names Celquat® H 100, Celquat® L 200.

Cationic film-forming and/or cationic fixing polymers are furthermore suitable which have at least one structural unit of formula (M-I) and at least one structural unit of formula (M-VI) and optionally at least one structural unit of formula (M-V)—

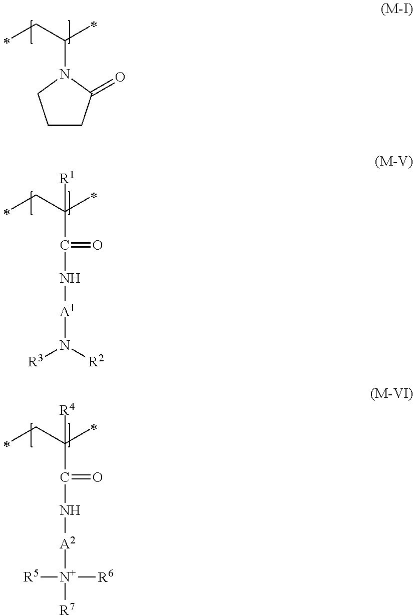

wherein
R¹ and R⁴ are independently a hydrogen atom or a methyl group,
A¹ and A² are independently an ethane-1,2-diyl, propane-1,3-diyl or butane-1,4-diyl group,
R², R³, R⁵ and R⁶ are independently a (C₁ to C₄) alkyl group, and
R⁷ is a (C₈ to C₃₀) alkyl group.

All possible physiologically tolerable anions, such as chloride, bromide, hydrogen sulfate, methyl sulfate, ethyl sulfate, tetrafluoroborate, phosphate, hydrogen phosphate, dihydrogen phosphate or p-toluenesulfonate, triflate, serve to offset the positive charge of the monomer (M-VI).

Commercially obtainable suitable compounds include—
copolymers of diethyl sulfate-quaternized dimethylaminoethyl methacrylate with vinyl pyrrolidone with the INCI name Polyquaternium-11, under the names Gafquat® 440, Gafquat® 734, Gafquat® 755 (all ISP) and Luviquat PQ 11 PN (BASF SE), and
copolymers of methacryloylaminopropyl lauryl dimethylammonium chloride with vinyl pyrrolidone and dimethylaminopropyl methacrylamide with the INCI name Polyquaternium-55, under the trade names Styleze® W-10, Styleze® W-20 (ISP).

Furthermore, cationic film-forming and/or cationic fixing polymers serving as film-forming and/or fixing polymers chosen from cationic polymers containing at least one structural unit having a permanently cationized nitrogen atom which can particularly preferably be used are those having at least one structural element of formula (M1)—

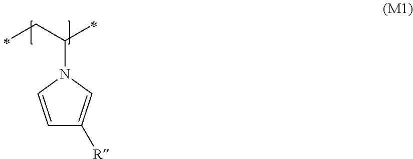

wherein
R″ is a (C₁ to C₄) alkyl group, particularly methyl, and
additionally having at least one further cationic and/or non-ionic structural element.

All possible physiologically tolerable anions, such as chloride, bromide, hydrogen sulfate, methyl sulfate, ethyl sulfate, tetrafluoroborate, phosphate, hydrogen phosphate, dihydrogen phosphate or p-toluenesulfonate, triflate, serve to offset the positive polymer charge of the component.

It is preferable for the powdery composition to contain as cationic film-forming and/or cationic fixing polymer at least one copolymer (b1) additionally containing in addition to at least one structural element of formula (M1) a structural element of formula (M-1)—

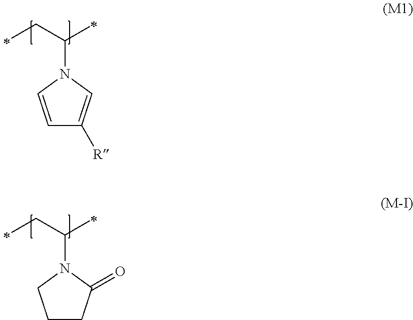

wherein
R″ is a (C₁ to C₄) alkyl group, particularly methyl.

All possible physiologically tolerable anions, such as chloride, bromide, hydrogen sulfate, methyl sulfate, ethyl sulfate, tetrafluoroborate, phosphate, hydrogen phosphate, dihydrogen phosphate or p-toluenesulfonate, triflate, serve to offset the positive polymer charge of copolymer (b1).

Most particularly preferred cationic film-forming and/or cationic fixing polymers as copolymers (b1) contain 10 to 30 mol %, preferably 15 to 25 mol % and particularly 20 mol % of structural units according to formula (M1) and 70 to 90 mol %, preferably 75 to 85 mol % and in particular 80 mol % of structural units according to formula (M-I).

It is particularly preferable here for copolymers (b1) to contain, in addition to polymer units resulting from the incorporation of the cited structural units according to formula (M1) and (M-I) in the copolymer, a maximum of 5 wt. %, preferably a maximum of 1 wt. %, of polymer units resulting from the incorporation of other monomers. Copolymers (b1) are preferably made up exclusively of structural units of formula (M1) with R″=methyl and (M-I) and can be described by the general formula (Poly1)—

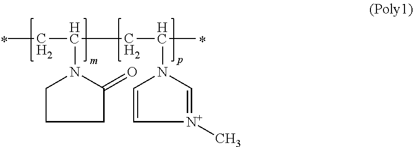

wherein m and p vary according to the molar mass of the polymer and are not intended to suggest that these are block copolymers. Rather, structural units of formula (M1) and formula (M-I) can be randomly distributed in the molecule.

If a chloride ion is used to offset the positive charge of the polymer of formula (Poly1), these N-methyl vinyl imidazole/vinyl pyrrolidone copolymers are referred to under INCI nomenclature as Polyquaternium-16 and are available for example from BASF under the trade names Luviquat® Style, Luviquat® FC 370, Luviquat® FC 550, Luviquat® FC 905 and Luviquat® HM 552.

If a methosulfate is used to offset the positive charge of the polymer of formula (Poly1), these N-methyl vinyl imidazole/vinyl pyrrolidone copolymers are referred to under INCI nomenclature as Polyquaternium-44 and are available for example from BASF under the trade name Luviquat® UltraCare.

Particularly preferred powdery compositions according to the invention contain a copolymer (b1), in particular of the formula (Poly1), having molar masses within a defined range. Agents according to the invention are preferred here in which the copolymer (b1) has a molar mass of 50 to 400 kDa, preferably 100 to 300 kDa, more preferably 150 to 250 kDa and in particular 190 to 210 kDa.

Powdery compositions according to the invention can also contain, in addition to copolymer(s) (b1) or in its or their place, copolymers (b2) which as additional structural units starting from copolymer (b1) have structural units of formula (M-II)—

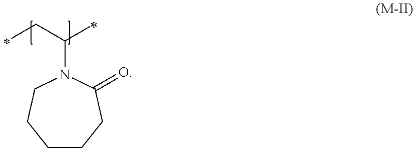

Further particularly preferred powdery compositions according to the invention contain as cationic film-forming and/or cationic fixing polymer at least one copolymer (b2) containing at least one structural unit according to formula (M1-a) and at least one structural unit according to formula (M-I) and at least one structural unit according to formula (M-II)—

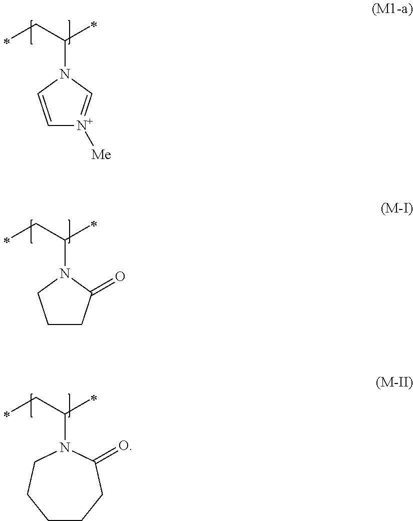

Here too it is particularly preferable for copolymers (b2) to contain, in addition to polymer units resulting from incorporation of the structural units according to formulae (M1-a), (M-I) and (M-II) in the copolymer, a maximum of 5 wt. %, preferably a maximum of 1 wt. %, of polymer units resulting from the incorporation of other monomers. Copolymers (b2) are preferably made up exclusively of structural units of formulae (M1-a), (M-I) and (M-II) and can be described by general formula (Poly2)—

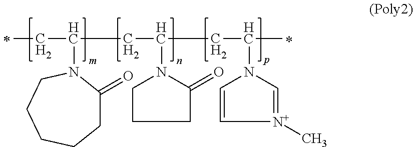

wherein m, n and p vary according to the molar mass of the polymer and are not intended to suggest that these are block copolymers. Rather, structural units of the formulae can be randomly distributed in the molecule.

All possible physiologically tolerable anions, such as for example chloride, bromide, hydrogen sulfate, methyl sulfate, ethyl sulfate, tetrafluoroborate, phosphate, hydrogen phosphate, dihydrogen phosphate or p-toluenesulfonate, triflate, serve to offset the positive polymer charge of the component (b2).

If a methosulfate is used to offset the positive charge of the polymer of formula (Poly2), such N-methyl vinyl imidazole/vinyl pyrrolidone/vinyl caprolactam copolymers are referred to under INCI nomenclature as Polyquaternium-46 and are available for example from BASF under the trade name Luviquat® Hold.

Most particularly preferred copolymers (b2) contain 1 to 20 mol. %, preferably 5 to 15 mol. % and in particular 10 mol % of structural units according to formula (M1-a) and 30 to 50 mol %, preferably 35 to 45 mol % and in particular 40 mol % of structural units according to formula (I) and 40 to 60 mol %, preferably 45 to 55 mol % and in particular 60 mol % of structural units according to formula (M-II).

Particularly preferred powdery compositions according to the invention contain a copolymer (b2) having molar masses within a defined range. Agents according to the invention are preferred here in which copolymer (b2) has a molar mass of 100 to 1000 kDa, preferably 250 to 900 kDa, more preferably 500 to 850 kDa and in particular 650 to 710 kDa.

As film-forming cationic and/or fixing cationic polymer powdery compositions according to the invention can also contain, in addition to copolymer(s) (b1) and/or (b2) or in its or their place, copolymers (b3) which as structural units have structural units of formulae (M1-a) and (I) as well as further structural units from vinyl imidazole units and further structural units from acrylamide and/or methacrylamide units.

Further particularly preferred powdery compositions according to the invention contain as cationic film-forming and/or cationic fixing polymer at least one copolymer (b3) having at least one structural unit according to formula (M1-a), at least one structural unit according to formula (M-I), at least one structural unit according to formula (M-VII) and at least one structural unit according to formula (M-VIII)—

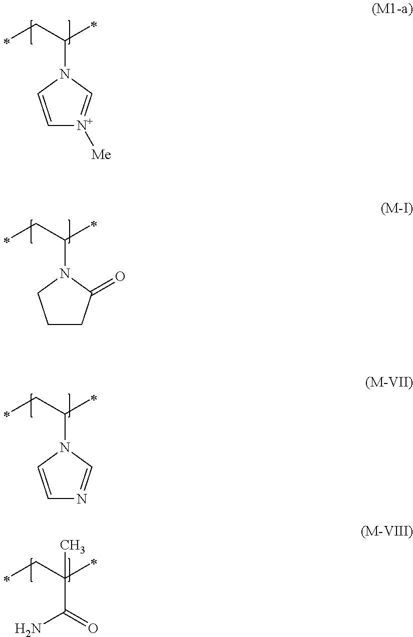

Here too it is particularly preferable for copolymers (b3) to contain, in addition to polymer units resulting from incorporation of structural units according to formulae (M1-a), (M-I), (M-VII) and (M-VIII) in the copolymer, a maximum of 5 wt. %, preferably a maximum of 1 wt. %, of polymer units resulting from the incorporation of other monomers. Copolymers (b3) are preferably made up exclusively of structural units of formula (M1-a), (M-I), (M-VII) and (M-VIII) and can be described by the general formula (Poly3)—

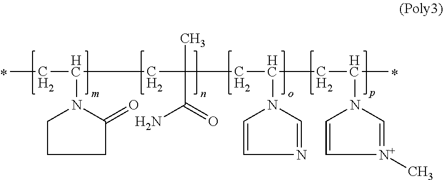

wherein m, n, o and p vary according to the molar mass of the polymer and are not intended to suggest that these are block copolymers. Rather, structural units of formulae (M1-a), (M-1), (M-VII) and (M-VIII) can be randomly distributed in the molecule.

All possible physiologically tolerable anions, such as chloride, bromide, hydrogen sulfate, methyl sulfate, ethyl sulfate, tetrafluoroborate, phosphate, hydrogen phosphate, dihydrogen phosphate or p-toluenesulfonate, triflate, serve to offset the positive polymer charge of component (b2).

If a methosulfate is used to offset the positive charge of the polymer of formula (Poly3), such N-methyl vinyl imidazole/vinyl pyrrolidone/vinyl imidazole/methacrylamide copolymers are referred to under INCI nomenclature as Polyquaternium-68 and are available for example from BASF under the trade name Luviquat® Supreme.

Most particularly preferred copolymers (b3) contain 1 to 12 mol. %, preferably 3 to 9 mol. %, and in particular 6 mol % of structural units according to formula (M1-a), 45 to 65 mol %, preferably 50 to 60 mol %, and in particular 55 mol % of structural units according to formula (M-I), 1 to 20 mol %, preferably 5 to 15 mol %, and in particular 10 mol % of structural units according to formula (M-VII), and 20 to 40 mol %, preferably 25 to 35 mol %, and in particular 29 mol % of structural units according to formula (M-VIII).

Particularly preferred agents according to the invention contain a copolymer (b3) having molar masses within a defined range. Preferred agents are those in which copolymer (b3) has a molar mass of 100 to 500 kDa, preferably 150 to 400 kDa, more preferably 250 to 350 kDa and in particular 290 to 310 kDa.

Of the additional film-forming cationic and/or fixing polymer chosen from cationic polymers having at least one structural element of formula (M1) above, the following are preferred:
Vinyl pyrrolidone/1-vinyl-3-methyl-1H-imidazolium chloride copolymers (such as that obtainable with the INCI name Polyquatemium-16 under the trade names Luviquat® Style, Luviquat® FC 370, Luviquat® FC 550, Luviquat® FC 905 and Luviquat® HM 552 (BASF SE)),
Vinyl pyrrolidone/1-vinyl-3-methyl-1H-imidazolium methyl sulfate copolymers (such as that obtainable with the INCI name Polyquatemium-44 under the trade name Luviquat® Care (BASF SE)),
Vinyl pyrrolidone/vinyl caprolactam/1-vinyl-3-methyl-1H-imidazolium terpolymer (such as that obtainable with the INCI name Polyquaternium-46 under the trade name Luviquat® Care or Luviquat® Hold (BASF SE)),
Vinyl pyrrolidone/methacrylamide/vinyl imidazole/1-vinyl-3-methyl-1H-imidazolium methyl sulfate copolymer (such as that obtainable with the INCI name Polyquaternium-68 under the trade name Luviquat® Supreme (BASF SE)),
   and mixtures of these polymers.

In a preferred embodiment powdery compositions according to the invention contain as film-forming and/or fixing polymer at least one film-forming non-ionic and/or fixing non-ionic polymer.

A non-ionic polymer according to the invention is a polymer which in a protic solvent under standard conditions substantially bears no structural units having permanently cationic or anionic groups needing to be offset by counterions to maintain electrical neutrality. Cationic groups include quaternized ammonium groups but not protonated amines. Anionic groups include carboxyl and sulfonic acid groups.

The film-forming non-ionic and/or fixing non-ionic polymers are preferably present in the agent in an amount from 0.1 wt. % to 20.0 wt. %, more preferably from 0.2 wt. % to 15.0 wt. %, most preferably from 0.5 wt. % to 5.0 wt. %, based on total weight of the powdery composition.

The film-forming non-ionic and/or fixing non-ionic polymers are preferably chosen from at least one polymer of the group formed from—
homopolymers and non-ionic copolymers of N-vinyl pyrrolidone,
non-ionic copolymers of isobutene.

Suitable polyvinyl pyrrolidones include commercial products such as Luviskol® K 90 or Luviskol® K 85 from BASF SE.

Suitable polyvinyl alcohols are sold for example under the trade names Elvanol® by Du Pont or Vinol® 523/540 by Air Products.

A suitable polyvinyl acetate is sold for example as an emulsion under the trade name Vinac® by Air Products.

Agents containing as film-forming non-ionic and/or fixing non-ionic polymer at least one polymer chosen from—
polyvinyl pyrrolidone,
copolymers of N-vinyl pyrrolidone and vinyl esters of carboxylic acids having 2 to 18 carbon atoms, particularly N-vinyl pyrrolidone and vinyl acetate,
copolymers of N-vinyl pyrrolidone and N-vinyl imidazole and methacrylamide,
copolymers of N-vinyl pyrrolidone and N-vinyl imidazole and acrylamide,
copolymers of N-vinyl pyrrolidone with N,N-di(C₁-C₄) alkylamino-(C₂-C₄)-alkylacrylamide, are most particularly preferred according to the invention.

Preferably, the molar ratio in the polymer of structural units contained from the N-vinyl pyrrolidone monomer to structural units contained from the vinyl acetate monomer is in the range from 20 to 80 to 80 to 20, particularly from 30 to 70 to 60 to 40.

Suitable copolymers of vinyl pyrrolidone and vinyl acetate are obtainable for example from BASF SE under the trademarks Luviskol® VA 37, Luviskol® VA 55, Luviskol® VA 64 and Luviskol® VA 73.

Further preferred powdery compositions according to the invention contain as the non-ionic film-forming and/or non-ionic fixing polymer at least one copolymer (n1) containing—

at least one further structural unit according to formula (M-I)

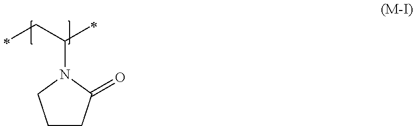

at least one further structural unit according to formula (M-VII)

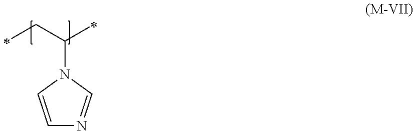

at least one further structural unit according to formula (M-VIII)

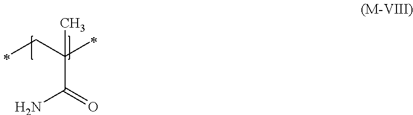

Here too it is particularly preferable for these copolymers to contain, in addition to polymer units resulting from the incorporation of the cited structural units according to formula (M1-a), (I), (VII) and (VIII) in the copolymer, a maximum of 5 wt. %, preferably a maximum of 1 wt. %, of polymer units resulting from the incorporation of other monomers. The copolymers (n1) are preferably made up exclusively of structural units of formula (M1-a), (I), (VII) and (VIII) and can be described by the general formula (Poly4)—

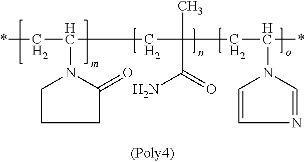

wherein m, n, o and p vary according to the molar mass of the polymer and are not intended to suggest that these are block copolymers. Rather, structural units of formulae (I), (VII) and (VIII) can be randomly distributed in the molecule.

A particularly preferred polymer is chosen from polymers with the INCI name VP/Methacrylamide/Vinyl Imidazole Copolymer obtainable, for example, under the trade name Luviset Clear from BASF SE.

Such powdery compositions containing at least one non-ionic film-forming and/or non-ionic fixing polymer are furthermore suitable according to the invention which comprise at least one structural unit of formula (M-I) and at least one structural unit of formula (M-III)—

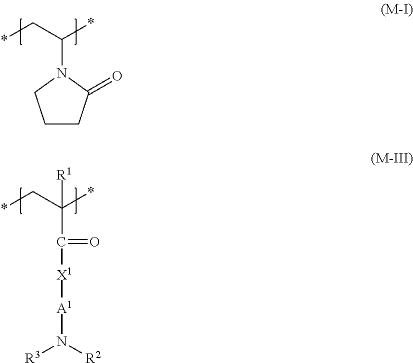

wherein
R¹ is a hydrogen atom or a methyl group,
X¹ is an oxygen atom or the group NH,
A¹ is an ethane-1,2-diyl, propane-1,3-diyl or butane-1,4-diyl group, and
R² and R³ are independently a (C₁ to C₄) alkyl group.

It is preferable in particular for the above non-ionic film-forming and/or non-ionic fixing polymer to be chosen from at least one polymer satisfying at least one or more of the following features:

R¹ is methyl,

X¹ is NH,

A¹ is ethane-1,2-diyl or propane-1,3-diyl, and

R² and R³ are independently methyl or ethyl (preferably methyl).

The non-ionic film-forming and/or non-ionic fixing polymer of this embodiment is preferably at least one polymer comprising at least one structural unit of formula (M-I) and at least one structural unit of formula (M-III-8)—

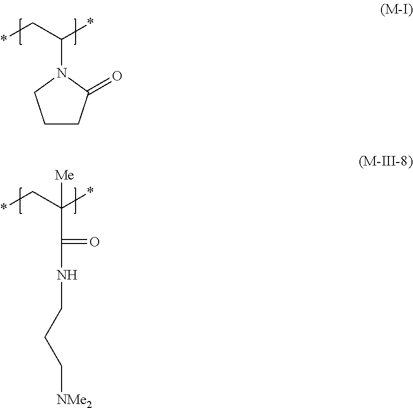

A most particularly preferred non-ionic film-forming and/or non-ionic fixing polymer of this embodiment is a copolymer of N-vinyl pyrrolidone and N,N-dimethylaminopropyl methacrylamide, which is sold for example with the INCI name VP/DMAPA Acrylates Copolymer under the trade name Styleze® CC 10 for example by ISP.

In a preferred embodiment compositions according to the invention contain as film-forming and/or fixing polymer at least one film-forming anionic and/or fixing anionic polymer.

An anionic polymer according to the invention is a polymer which in a protic solvent under standard conditions bears structural units having anionic groups needing to be offset by counterions to maintain electrical neutrality and no structural units having permanently cationic or cationizable groups. Anionic groups include carboxyl and sulfonic acid groups.

Anionic film-forming and/or anionic fixing polymers are preferably included in the agent according to the invention in an amount from 0.1 wt. % to 20.0 wt. % particularly preferably from 0.2 wt. % to 15.0 wt. %, most particularly preferably from 0.5 wt. % to 10.0 wt. %, based on total weight of the composition.

Preferably, the anionic film-forming and/or anionic fixing polymers contain at least one structural unit of formula (S1) chosen from at least one structural unit of formulae (S1-1) to (S1-5)—

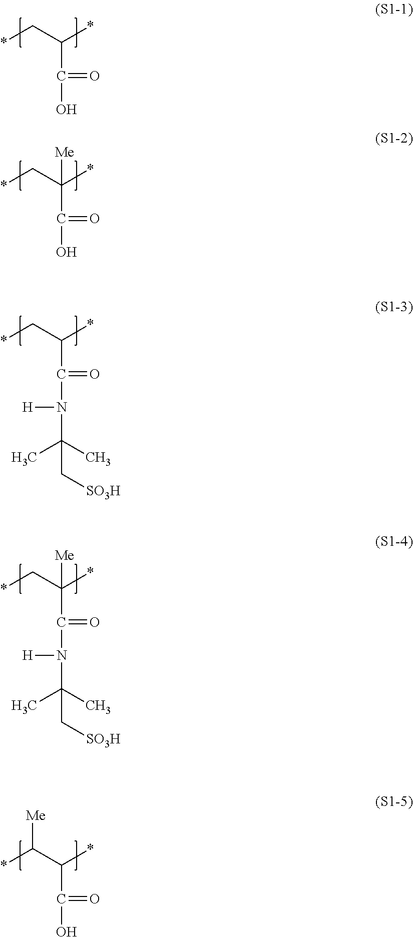

Preferably the anionic film-forming and/or anionic fixing polymers contain, in addition to at least one structural unit of formulae (S1-1) to (S1-5), at least one structural unit of formula (S2) chosen from at least one structural unit of formulae (S2-1) to (S2-8)—

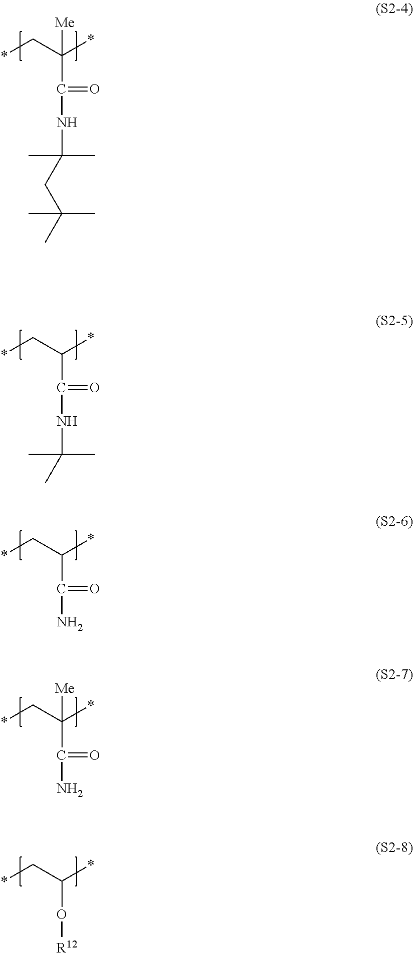

wherein R¹² is a (C₂ to C₁₂) acyl group (particularly acetyl or neodecanoyl).

In a preferred embodiment such powdery compositions are preferred which contain as film-forming and/or fixing polymer in particle form at least one polymer containing at least one structural unit of formula (S1-5) and at least one structural unit of formula (S2-8)—

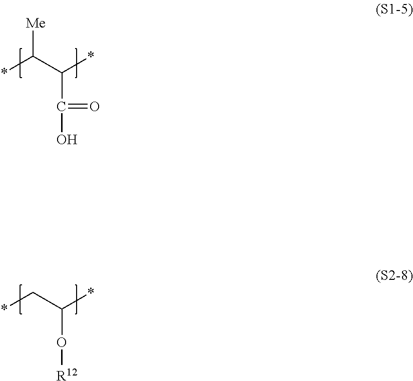

wherein R¹² is a (C₂ to C₁₂) acyl group (particularly acetyl and/or neodecanoyl).

Particularly preferred polymers of this type are chosen from at least one polymer of the group—

copolymers of vinyl acetate and crotonic acid,

copolymers of vinyl propionate and crotonic acid,

copolymers of vinyl neodecanoate, vinyl acetate and crotonic acid.

Such copolymers are available, for example, from Clariant under the trade name Aristoflex A 60 (INCI name: VA/Crotonates Copolymer) in an isopropanol/water blend (60 wt. % active substance), from BASF under the trade name Luviset CA 66 (vinyl acetate/crotonic acid copolymer 90:10, INCI name VA/Crotonates Copolymer), and from National Starch under the trade name Resyn 28-2942 and Resyn 28-2930 (INCI name: VA/Crotonates/Vinyl Neodecanoate Copolymer).

In a preferred embodiment such powdery compositions contain as anionic film-forming and/or anionic fixing polymer at least one polymer containing at least one structural unit of formula (S1-1) and at least one structural unit of formula (S2-5)—

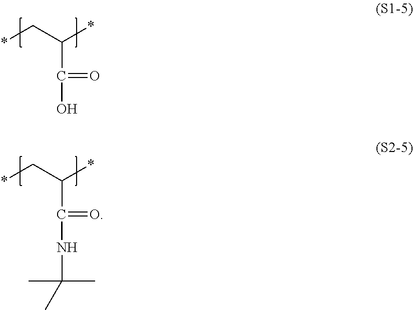

It is particularly preferable here for the film-forming and/or fixing polymer in particle form to contain, in addition to the above structural units of formulae (S1-1) and (S2-5), at least one structural unit of formula (S3)—

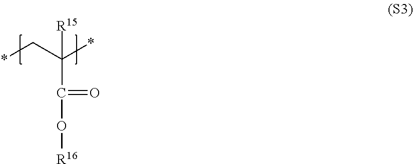

wherein
R¹⁵ is a hydrogen atom or a methyl group, and
R¹⁶ is a (C₁ to C₄) alkyl group (particularly a methyl group or an ethyl group).

Particularly preferred polymers of this type are chosen from at least one polymer from the group copolymers of acrylic acid and ethyl acrylate and N-tert-butyl acrylamide. Such copolymers are provided, for example, by BASF under the trade name Ultrahold® Strong (INCI name: Acrylates/t-Butylacrylamide Copolymer, white, pourable granules) or Ultrahold® 8 (INCI name: Acrylates/t-Butylacrylamide Copolymer, white, pourable granules).

In one embodiment, such agents are preferred which contain as anionic film-forming and/or anionic fixing polymer at least one polymer containing at least one structural unit of formula (S1-3) and at least one structural unit of formula (S2-6)—

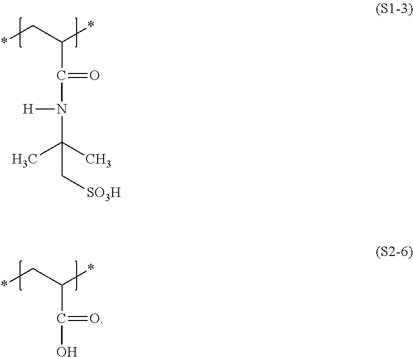

Preferred polymers (b) of this type are chosen from at least one polymer of the group—

copolymers of 2-acrylamido-2-methylpropane sulfonic acid and acrylamide,

copolymers of 2-acrylamido-2-methylpropane sulfonic acid, acrylamide and acrylic acid,

copolymers of 2-acrylamido-2-methylpropane sulfonic acid, acrylamide and methacrylic acid.

Polymers of this type are sold, for example, in an invert isohexadecane emulsion by Seppic under the trade name Sepigel® 305 (INCI name: Polyacrylamide, C13-14 Isoparaffin, Laureth-7) or Simulgel® 600 (INCI name: Acrylamide/Acryloyldimethyltaurate Copolymer, Isohexadecane, Polysorbate-80).

A particularly preferred agent, contains as polymer a copolymer (b5).

These copolymers (b5) can be described by the general formula—

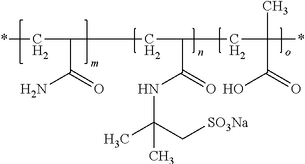

wherein m, n and o vary according to the molar mass of the polymer and are not intended to suggest that these are block copolymers. Rather, the structural units can be randomly distributed in the molecule.

Particularly preferred agents according to the invention have the characterizing feature that copolymer (b1) has a molar mass of 50 to 500 kDa, preferably 100 to 450 kDa, more preferably 150 to 400 kDa, and particularly 200 to 300 kDa.

Copolymers of acrylamide with methacrylic acid and acryloyldimethyltaurate are obtainable, for example, under the trade name Acudyne® SCP (Rohm & Haas).

In a further embodiment, powdery compositions according to the invention contain as film-forming and/or fixing polymer at least one film-forming amphoteric and/or fixing amphoteric polymer.

An amphoteric polymer according to the invention is a polymer which in a protic solvent under standard conditions bears structural units having anionic groups needing to be offset by counterions to maintain electrical neutrality, and additionally structural units having groups which can be cationized by protonation but is free from permanently cationized groups. Anionic groups include carboxyl and sulfonic acid groups. Permanently cationized nitrogen atoms refer to nitrogen atoms bearing a positive charge and thus forming a quaternary ammonium compound.

Amphoteric film-forming and/or amphoteric fixing polymers are preferably present in the powdery composition in an amount from 0.1 wt. % to 20.0 wt. %, more preferably from 0.2 wt. % to 15.0 wt. %, most preferably from 0.5 wt. % to 10.0 wt. %, based on total weight of the composition according to the invention.

It is suitable for the amphoteric film-forming and/or amphoteric fixing polymer to contain at least one structural unit of formula (S1) chosen from at least one structural unit of formulae (S1-1) to (S1-5)—

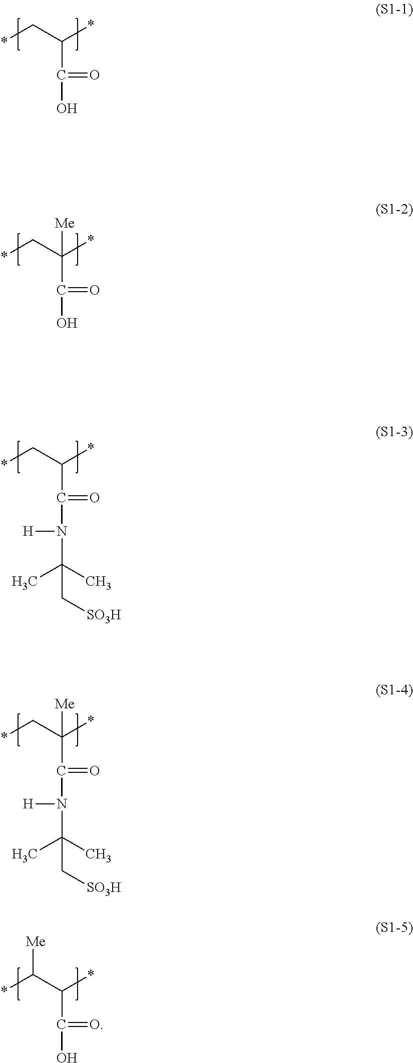

It is suitable for the amphoteric film-forming and/or amphoteric fixing polymers additionally to contain, in addition to at least one structural unit of formulae (S1-1) to (S1-5), at least one structural unit of formula (S2) chosen from at least one structural unit of formulae (S2-9) to (S2-15)—

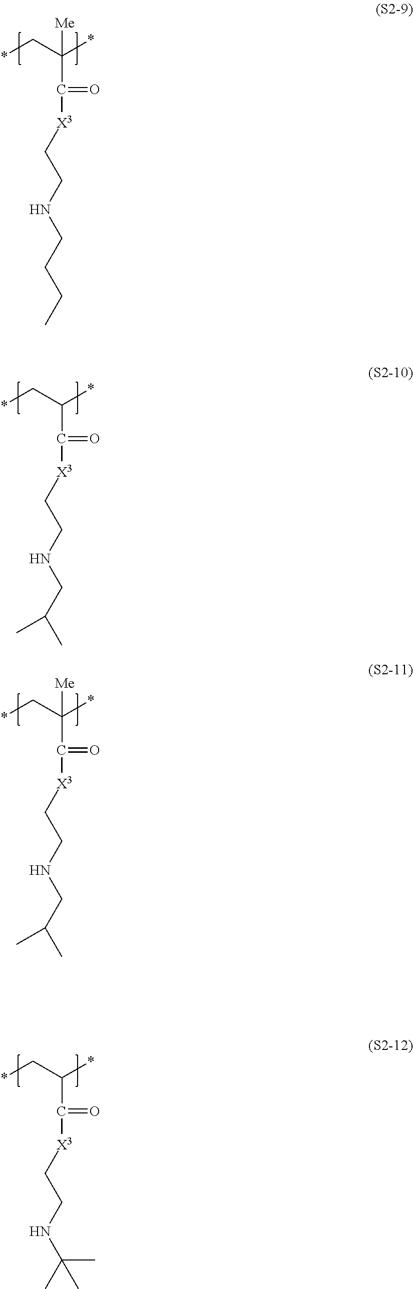

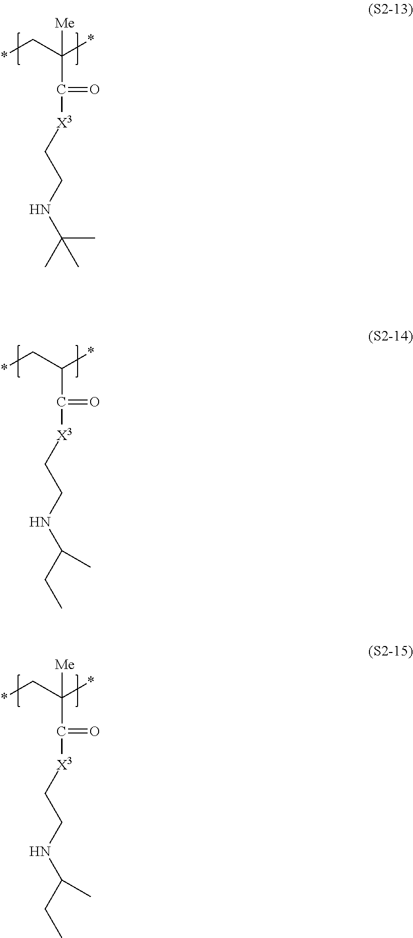

wherein X³ is an oxygen atom or an NH group.

It is further suitable for the amphoteric film-forming and/or amphoteric fixing polymers to additionally contain, in addition to at least one structural unit of formulae (S1-1) to (S1-5) and at least one structural unit of formulae (S2-9) to (S2-15), at least one structural unit of formulae (S2-1) to (S2-8)—

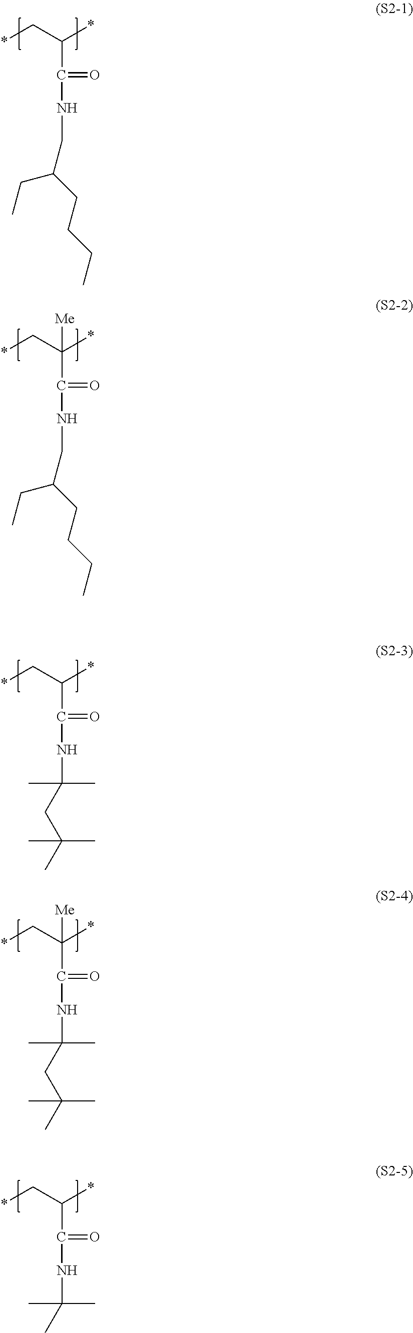

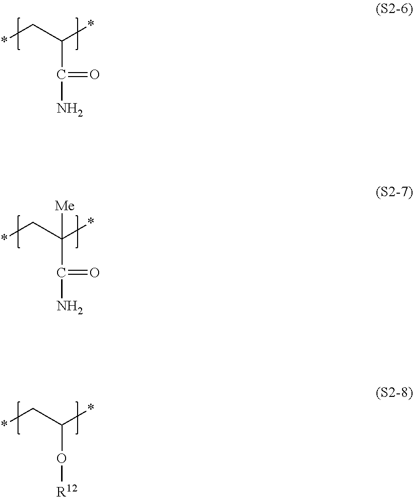

wherein R¹² is a (C₂ to C₁₂) acyl group (particularly acetyl or neodecanoyl).

In a further embodiment, the powdery composition contains at least one amphoteric film-forming and/or amphoteric fixing polymer containing at least one structural unit of formula (S1-1), at least one structural unit of formula (S2-3) and at least one structural unit of formula (S2-16) (chosen from the above formulae (S2-5) to (S2-12) with the proviso that X³ is an oxygen atom)—

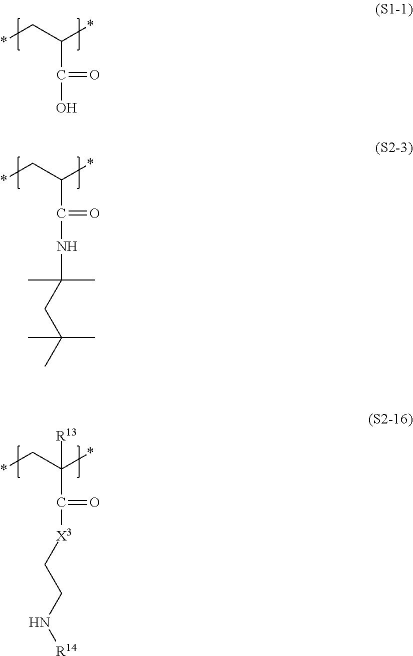

wherein
X³ is an oxygen atom or an NH group,
R¹³ is a hydrogen atom or a methyl group, and
R¹⁴ is an alkyl group having 4 carbon atoms (particularly n-butyl, sec-butyl, isobutyl or tert-butyl).

It is also particularly suitable for the amphoteric film-forming and/or anionic fixing polymers to additionally contain, in addition to the above structural units of formulae (S1-1), (S2-3) and (S2-16), at least one structural unit of formula (S3)—

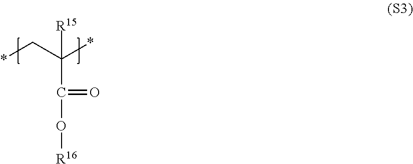

wherein
R¹⁵ is a hydrogen atom or a methyl group, and
R¹⁶ is a (C₁ to C₄) alkyl group (particularly methyl or ethyl).

Preferred polymers of this type are chosen from copolymers of acrylic acid, (C₁ to C₄) alkyl acrylate, N—(C₄ alkyl)aminoethyl methacrylate and N—(C₈ alkyl)acrylamide.

An example of a film-forming and/or fixing polymer in particle form which can particularly preferably be used in this embodiment is the polymer obtainable under the trade name Amphomer® from National Starch with the INCI name Octylacrylamide/Acrylates/Butylaminoethylmethacrylate Copolymer.

Powdery compositions additionally containing at least one monosaccharide and/or at least one disaccharide bring about in particular improved properties of the resulting hairstyle.

Both monosaccharides and disaccharides such as cane sugar, lactose and raffinose can be used. Use of monosaccharides is preferred. Of the monosaccharides, compounds containing 5 or 6 carbon atoms are preferred.

Suitable pentoses and hexoses include ribose, arabinose, xylose, lyxose, allose, altrose, glucose, mannose, gulose, idose, galactose, talose, fucose and fructose. Arabinose, glucose, galactose and fructose are preferably used carbohydrates. Glucose is most preferably used, which is suitable both in the D-(+)- or L-(−)-configuration or as a racemate.

Mono- or disaccharides are preferably contained in the powdery compositions in an amount from 0.1 to 8 wt. %, particularly 1 to 5 wt. %, based on total powdery composition.

Similar effects can also be achieved by adding at least one sugar alcohol derived from pentoses and/or from hexoses. Preferred sugar alcohols are sorbitol, mannitol and dulcitol, sorbitol being particularly preferably used.

Sugar alcohols derived from pentoses and/or from hexoses are preferably present in powdery compositions according to the invention in an amount from 0.1 to 8 wt. %, particularly 1 to 5 wt %, based on total weight of the powdery composition.

Preferred powdery compositions additionally contain at least one alkalizing agent.

Useful alkalizing agents are preferably chosen from basic amino acids, alkali hydroxides, alkanol amines, alkali metal metasilicates, urea, morpholine, N-methyl glucamine, alkali phosphates and alkali hydrogen phosphates. Preferred alkali metal ions are lithium, sodium, potassium, particularly sodium or potassium.

Basic amino acids which can be used as alkalizing agents are preferably chosen from L-arginine, D-arginine, D,L-arginine, L-histidine, D-histidine, D,L-histidine, L-lysine, D-lysine, D,L-lysine. L-arginine, D-arginine and D,L-arginine are preferably used as an alkalizing agent within the meaning of the invention.

Alkali hydroxides which can be used as the alkalizing agent according to the invention are preferably chosen from sodium hydroxide and potassium hydroxide.

Useful alkanol amines as the alkalizing agent are preferably chosen from primary amines having a C₂-C₆ alkyl parent substance bearing at least one hydroxyl group. Particularly preferred alkanol amines are chosen from 2-aminoethan-1-ol (monoethanolamine), triethanolamine, 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, 2-amino-2-methylpropane-1,3-diol. Most particularly preferred alkanol amines according to the invention are selected from the group comprising triethanolamine, 2-aminoethan-1-ol, 2-amino-2-methylpropan-1-ol and 2-amino-2-methylpropane-1,3-diol.

The alkalizing agent is particularly preferably chosen from triethanolamine, 2-aminoethanol, 2-amino-2-methylpropan-1-ol, 2-amino-2-methylpropane-1,3-diol, potassium hydroxide, L-arginine, D-arginine, D,L-arginine.

It is furthermore preferable according to the invention for the powdery composition to additionally contain at least one extract from algae. This algae extract is preferably obtained by extraction from red algae and/or from brown algae. The algae extract with the CAS number 92128-82-0 is most particularly preferred. This is sold, for example, in the commercial product Fucosorb® by Sensient/LCW.

Powdery compositions according to the invention preferably contain 0.1 to 4 wt. % of algae extract based on total weight of the powdery composition.

The following embodiments (A) to (X) represent most particularly preferred embodiments of the powdery composition according to the invention:

(A): A powdery composition for temporarily shaping human hair, containing
i) 50 to 90 wt. % of water,
ii) 0.5 wt. % to 10 wt. % of at least one liquid having a boiling point higher than 150° C. at a pressure of 1013 mbar, chosen from at least one (C₃-C₈) alcohol having at least two hydroxyl groups,
iii) carboxymethyl starch, and
iv) metal oxide particles coated on the surface with fluorine-containing organic groups.

(B): A powdery composition for temporarily shaping human hair, containing
i) 50 to 90 wt. % of water,
ii) 0.5 wt. % to 10 wt. % of at least one liquid having a boiling point higher than 150° C. at a pressure of 1013 mbar, chosen from at least one compound of glycerol, 1,3-butylene glycol, 1,4-butylene glycol, 1,2-pentanediol, 2-methyl-2,4-dihydroxypentane and 2-ethyl-1,3-hexanediol,
iii) carboxymethyl starch, and
iv) metal oxide particles coated on the surface with fluorine-containing organic groups.

(C): A powdery composition for temporarily shaping human hair, containing
i) 50 to 90 wt. % of water,
ii) 0.5 wt. % to 10 wt. % of glycerol,
iii) carboxymethyl starch, and
iv) metal oxide particles coated on the surface with fluorine-containing organic groups.

(D): A powdery composition for temporarily shaping human hair, containing
i) 50 to 90 wt. % of water,
ii) 0.5 wt. % to 10 wt. % of at least one liquid having a boiling point higher than 150° C. at a pressure of 1013 mbar, chosen from at least one (C₃-C₈) alcohol having at least two hydroxyl groups,
iii) carboxymethyl starch, and
iv) 5 wt. % to 25 wt. % of metal oxide particles coated on the surface with fluorine-containing organic groups.

(E): A powdery composition for temporarily shaping human hair, containing
i) 50 to 90 wt. % of water,
ii) 0.5 wt. % to 10 wt. % of at least one liquid having a boiling point higher than 150° C. at a pressure of 1013 mbar, chosen from at least one of glycerol, 1,3-butylene glycol, 1,4-butylene glycol, 1,2-pentanediol, 2-methyl-2,4-dihydroxypentane and 2-ethyl-1,3-hexanediol,
iii) carboxymethyl starch, and
iv) 5 wt. % to 25 wt. % of metal oxide particles coated on the surface with fluorine-containing organic groups.

(F): A powdery composition for temporarily shaping human hair, containing
i) 50 to 90 wt. % of water,
ii) 0.5 wt. % to 10 wt. % of glycerol,
iii) carboxymethyl starch, and
iv) 5 wt. % to 25 wt. % of metal oxide particles coated on the surface with fluorine-containing organic groups.

(G): A powdery composition for temporarily shaping human hair, containing
i) 50 to 90 wt. % of water,
ii) 0.5 wt. % to 10 wt. % of at least one further liquid having a boiling point higher than 150° C. at a pressure of 1013 mbar, chosen from at least one (C₃ to C₈) alcohol having at least two hydroxyl groups,
iii) carboxymethyl starch,
iv) metal oxide particles coated on the surface with fluorine-containing organic groups,
v) metal oxide particles coated on the surface by reaction with at least one reagent of formula (II),

(R¹O)ₙ(CH₃)₃₋ₙSi—R² (II)

wherein

R¹ is methyl or ethyl,

R² is a (C₁ to C₁₄) alkyl group, particularly a (C₆ to C₁₂) alkyl group, and

n is 1, 2 or 3.

(H): A powdery composition for temporarily shaping human hair, containing
i) 50 to 90 wt. % of water,
ii) 0.5 wt. % to 10 wt. % of at least one further liquid having a boiling point higher than 150° C. at a pressure of 1013 mbar, chosen from at least one compound from glycerol, 1,3-butylene glycol, 1,4-butylene glycol, 1,2-pentanediol, 2-methyl-2,4-dihydroxypentane and 2-ethyl-1,3-hexanediol,
iii) carboxymethyl starch,
iv) metal oxide particles coated on the surface with fluorine-containing organic groups,
v) metal oxide particles coated on the surface by reaction with at least one reagent of formula (II),

(R¹O)ₙ(CH₃)₃₋ₙSi—R² (II)

wherein

R¹ is methyl or ethyl,

R² is a (C₁ to C₁₄) alkyl group, particularly a (C₆ to C₁₂) alkyl group, and

n is 1, 2 or 3.

(I): A powdery composition for temporarily shaping human hair, containing
i) 50 to 90 wt. % of water,
ii) 0.5 wt. % to 10 wt. % of glycerol,
iii) carboxymethyl starch,
iv) metal oxide particles coated on the surface with fluorine-containing organic groups,
v) metal oxide particles coated on the surface by reaction with at least one reagent of formula (II),

(R¹O)ₙ(CH₃)₃₋ₙSi—R² (II)

wherein

R¹ is methyl or ethyl,

R² is a (C₁ to C₁₄) alkyl group, particularly a (C₆ to C₁₂) alkyl group, and

n is 1, 2 or 3.

(J): A powdery composition for temporarily shaping human hair, containing
i) 50 to 90 wt. % of water,
ii) 0.5 wt. % to 10 wt. % of at least one further liquid having a boiling point higher than 150° C. at a pressure of 1013 mbar, chosen from at least one (C₃ to C₈) alcohol having at least two hydroxyl groups,
iii) carboxymethyl starch,
iv) metal oxide particles coated on the surface with at least one reagent of formula (I),

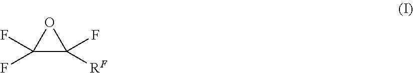

wherein

R^{F} is a perfluoro-(C₁ to C₄) alkyl group, particularly trifluoromethyl,
v) metal oxide particles coated on the surface by reaction with at least one reagent of formula (II),

(R¹O)ₙ(CH₃)₃₋ₙSi—R² (II)

wherein

R¹ is methyl or ethyl,

R² is a (C₁ to C₁₄) alkyl group, particularly a (C₆ to C₁₂) alkyl group, and

n is 1, 2 or 3.

(K): A powdery composition for temporarily shaping human hair, containing
i) 50 to 90 wt. % of water,
ii) 0.5 wt. % to 10 wt. % of at least one further liquid having a boiling point higher than 150° C. at a pressure of 1013 mbar, chosen from at least one compound from glycerol, 1,3-butylene glycol, 1,4-butylene glycol, 1,2-pentanediol, 2-methyl-2,4-dihydroxypentane and 2-ethyl-1,3-hexanediol,
iii) carboxymethyl starch,
iv) metal oxide particles coated on the surface with at least one reagent of formula (I),

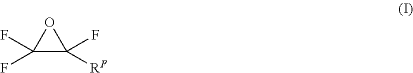

wherein

R^{F} is a perfluoro-(C₁ to C₄) alkyl group, particularly trifluoromethyl,
v) metal oxide particles coated on the surface by reaction with at least one reagent of formula (II),

(R¹O)ₙ(CH₃)₃₋ₙSi—R² (II)

wherein

R¹ is methyl or ethyl,

R² is a (C₁ to C₁₄) alkyl group, particularly a (C₆ to C₁₂) alkyl group, and

n is 1, 2 or 3.

(L): A powdery composition for temporarily shaping human hair, containing
i) 50 to 90 wt. % of water,
ii) 0.5 wt. % to 10 wt. % of glycerol,
iii) carboxymethyl starch,
iv) metal oxide particles coated on the surface with at least one reagent of formula (I),

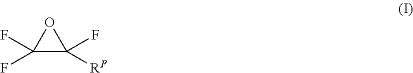

wherein

R^{F} is a perfluoro-(C₁ to C₄) alkyl group, particularly trifluoromethyl,
v) metal oxide particles coated on the surface by reaction with at least one reagent of formula (II),

(R¹O)ₙ(CH₃)₃₋ₙSi—R² (II)

wherein

R¹ is methyl or ethyl,

R² is a (C₁ to C₁₄) alkyl group, particularly a (C₆ to C₁₂) alkyl group, and

n is 1, 2 or 3.

(M): A powdery composition for temporarily shaping human hair, containing
i) 50 to 90 wt. % of water,
ii) 0.5 wt. % to 10 wt. % of at least one further liquid having a boiling point higher than 150° C. at a pressure of 1013 mbar, chosen from at least one (C₃ to C₈) alcohol having at least two hydroxyl groups,
iii) carboxymethyl starch,
iv) metal oxide particles coated on the surface with at least one reagent of formula (Ia),

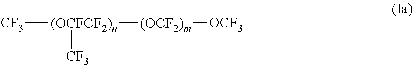

wherein the sum of n+m is a whole number from 20 to 80,
v) metal oxide particles coated on the surface by reaction with at least one reagent of formula (II),

(R¹O)ₙ(CH₃)₃₋ₙSi—R² (II)

wherein

R¹ is methyl or ethyl,

R² is a (C₁ to C₁₄) alkyl group, particularly a (C₆ to C₁₂) alkyl group, and

n is 1, 2 or 3.

(N): A powdery composition for temporarily shaping human hair, containing
i) 50 to 90 wt. % of water,
ii) 0.5 wt. % to 10 wt. % of at least one further liquid having a boiling point higher than 150° C. at a pressure of 1013 mbar, chosen from at least one compound from glycerol, 1,3-butylene glycol, 1,4-butylene glycol, 1,2-pentanediol, 2-methyl-2,4-dihydroxypentane and 2-ethyl-1,3-hexanediol,
iii) carboxymethyl starch,
iv) metal oxide particles coated on the surface with at least one reagent of formula (Ia),

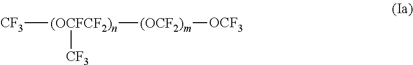

wherein the sum of n+m is a whole number from 20 to 80,
v) metal oxide particles coated on the surface by reaction with at least one reagent of formula (II),

(R¹O)ₙ(CH₃)₃₋ₙSi—R² (II)

wherein

R¹ is methyl or ethyl,

R² is a (C₁ to C₁₄) alkyl group, particularly a (C₆ to C₁₂) alkyl group, and

n is 1, 2 or 3.

(O): A powdery composition for temporarily shaping human hair, containing
i) 50 to 90 wt. % of water,
ii) 0.5 wt. % to 10 wt. % of glycerol,
iii) carboxymethyl starch,
iv) metal oxide particles coated on the surface with at least one reagent of formula (Ia),

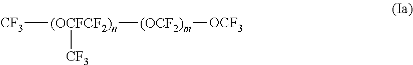

wherein the sum of n+m is a whole number from 20 to 80,
v) metal oxide particles coated on the surface by reaction with at least one reagent of formula (II),

(R¹O)ₙ(CH₃)₃₋ₙSi—R² (II)

wherein

R¹ is methyl or ethyl,

R² is a (C₁ to C₁₄) alkyl group, particularly a (C₆ to C₁₂) alkyl group, and

n is 1, 2 or 3.

(P): A powdery composition for temporarily shaping human hair comprising core-shell particles whose shell contains metal oxide particles coated on the surface with fluorine-containing organic groups and whose core comprises a liquid, aqueous phase, wherein the powdery composition contains
i) 50 to 90 wt. % of water,
ii) 0.5 wt. % to 10 wt. % of at least one further liquid having a boiling point higher than 150° C. at a pressure of 1013 mbar, chosen from at least one (C₃ to C₈) alcohol having at least two hydroxyl groups, and
iii) carboxymethyl starch.

(Q): A powdery composition for temporarily shaping human hair comprising core-shell particles whose shell contains metal oxide particles coated on the surface with fluorine-containing organic groups and whose core comprises a liquid, aqueous phase, wherein the powdery composition contains
i) 50 to 90 wt. % of water,
ii) 0.5 wt. % to 10 wt. % of at least one further liquid having a boiling point higher than 150° C. at a pressure of 1013 mbar, chosen from at least one compound from glycerol, 1,3-butylene glycol, 1,4-butylene glycol, 1,2-pentanediol, 2-methyl-2,4-dihydroxypentane and 2-ethyl-1,3-hexanediol, and
iii) carboxymethyl starch.

(R): A powdery composition for temporarily shaping human hair comprising core-shell particles whose shell contains metal oxide particles coated on the surface with fluorine-containing organic groups and whose core comprises a liquid, aqueous phase, wherein the powdery composition contains
i) 50 to 90 wt. % of water,
ii) 0.5 wt. % to 10 wt. % of glycerol, and
iii) carboxymethyl starch.

(S): A powdery composition for temporarily shaping human hair comprising core-shell particles whose shell contains 5 wt. % to 25 wt. % of metal oxide particles coated on the surface with fluorine-containing organic groups and whose core comprises a liquid, aqueous phase, wherein the powdery composition contains
i) 50 to 90 wt. % of water,
ii) 0.5 wt. % to 10 wt. % of at least one further liquid having a boiling point higher than 150° C. at a pressure of 1013 mbar, chosen from at least one (C₃ to C₈) alcohol having at least two hydroxyl groups, and
iii) carboxymethyl starch.

(T): A powdery composition for temporarily shaping human hair comprising core-shell particles whose shell contains 5 wt. % to 25 wt. % of metal oxide particles coated on the surface with fluorine-containing organic groups and whose core comprises a liquid, aqueous phase, wherein the powdery composition contains
i) 50 to 90 wt. % of water,
ii) 0.5 wt. % to 10 wt. % of at least one further liquid having a boiling point higher than 150° C. at a pressure of 1013 mbar, chosen from at least one compound from glycerol, 1,3-butylene glycol, 1,4-butylene glycol, 1,2-pentanediol, 2-methyl-2,4-dihydroxypentane and 2-ethyl-1,3-hexanediol, and
iii) carboxymethyl starch.

(U): A powdery composition for temporarily shaping human hair comprising core-shell particles whose shell contains 5 wt. % to 25 wt. % of metal oxide particles coated on the surface with fluorine-containing organic groups and whose core comprises a liquid, aqueous phase, wherein the powdery composition contains
i) 50 to 90 wt. % of water,
ii) 0.5 wt. % to 10 wt. % of glycerol, and
iii) carboxymethyl starch.

(V): A powdery composition for temporarily shaping human hair comprising core-shell particles whose shell contains
(a) metal oxide particles coated on the surface with fluorine-containing organic groups, and
(b) metal oxide particles coated on the surface by reaction with at least one reagent of formula (II),

(R¹O)ₙ(CH₃)₃₋ₙSi—R² (II)

wherein

R¹ is methyl or ethyl,

R² is a (C₁ to C₁₄) alkyl group, particularly a (C₆ to C₁₂) alkyl group, and

n is 1, 2 or 3,

and whose core comprises a liquid, aqueous phase, wherein the powdery composition contains
i) 50 to 90 wt. % of water,
ii) 0.5 wt. % to 10 wt. % of at least one further liquid having a boiling point higher than 150° C. at a pressure of 1013 mbar, chosen from at least one (C₃ to C₈) alcohol having at least two hydroxyl groups, and
iii) carboxymethyl starch.

(W): A powdery composition for temporarily shaping human hair comprising core-shell particles whose shell contains
(a) metal oxide particles coated on the surface with fluorine-containing organic groups, and
(b) metal oxide particles coated on the surface by reaction with at least one reagent of formula (II),

(R¹O)ₙ(CH₃)₃₋ₙSi—R² (II)

wherein

R¹ is methyl or ethyl,

R² is a (C₁ to C₁₄) alkyl group, particularly a (C₆ to C₁₂) alkyl group, and

n is 1, 2 or 3,

and whose core comprises a liquid, aqueous phase, wherein the powdery composition contains
i) 50 to 90 wt. % of water,
ii) 0.5 wt. % to 10 wt. % of at least one further liquid having a boiling point higher than 150° C. at a pressure of 1013 mbar, chosen from at least one compound from glycerol, 1,3-butylene glycol, 1,4-butylene glycol, 1,2-pentanediol, 2-methyl-2,4-dihydroxypentane and 2-ethyl-1,3-hexanediol, and
iii) carboxymethyl starch.

(X): A powdery composition for temporarily shaping human hair comprising core-shell particles whose shell contains
(a) metal oxide particles coated on the surface with fluorine-containing organic groups, and
(b) metal oxide particles coated on the surface by reaction with at least one reagent of formula (II),

(R¹O)ₙ(CH₃)₃₋ₙSi—R² (II)

wherein

R¹ is methyl or ethyl,

R² is a (C₁ to C₁₄) alkyl group, particularly a (C₆ to C₁₂) alkyl group, and

n is 1, 2 or 3,

and whose core comprises a liquid, aqueous phase, wherein the powdery composition contains

i) 50 to 90 wt. % of water,

ii) 0.5 wt. % to 10 wt. % of glycerol, and

iii) carboxymethyl starch.

(Y): A powdery composition for temporarily shaping human hair comprising core-shell particles whose shell contains
(a) metal oxide particles coated on the surface by reaction with at least one reagent of formula (I),

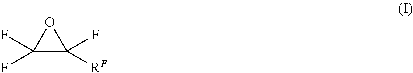

wherein R^{F} is a perfluoro-(C₁ to C₄) alkyl group, particularly trifluoromethyl,
(b) metal oxide particles coated on the surface by reaction with at least one reagent of formula (II),

(R¹O)ₙ(CH₃)₃₋ₙSi—R² (II)

wherein

R¹ is methyl or ethyl,

R² is a (C₁ to C₁₄) alkyl group, particularly a (C₆ to C₁₂) alkyl group, and

n is 1, 2 or 3,

and whose core comprises a liquid, aqueous phase, wherein the powdery composition contains
i) 50 to 90 wt. % of water,
ii) 0.5 wt. % to 10 wt. % of at least one further liquid having a boiling point higher than 150° C. at a pressure of 1013 mbar, chosen from at least one (C₃ to C₈) alcohol having at least two hydroxyl groups, and
iii) carboxymethyl starch.

(Z): A powdery composition for temporarily shaping human hair comprising core-shell particles whose shell contains
(a) metal oxide particles coated on the surface by reaction with at least one reagent of formula (I),

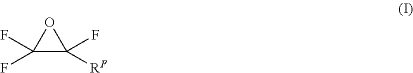

wherein R^{F} is a perfluoro-(C₁ to C₄) alkyl group, particularly trifluoromethyl,
(b) metal oxide particles coated on the surface by reaction with at least one reagent of formula (II),

(R¹O)ₙ(CH₃)₃₋ₙSi—R² (II)

wherein

R¹ is methyl or ethyl,

R² is a (C₁ to C₁₄) alkyl group, particularly a (C₆ to C₁₂) alkyl group, and

n is 1, 2 or 3,

and whose core comprises a liquid, aqueous phase, wherein the powdery composition contains
i) 50 to 90 wt. % of water,
ii) 0.5 wt. % to 10 wt. % of at least one further liquid having a boiling point higher than 150° C. at a pressure of 1013 mbar, chosen from at least one compound from glycerol, 1,3-butylene glycol, 1,4-butylene glycol, 1,2-pentanediol, 2-methyl-2,4-dihydroxypentane and 2-ethyl-1,3-hexanediol, and
iii) carboxymethyl starch.

(AA): A powdery composition for temporarily shaping human hair comprising core-shell particles whose shell contains
(a) metal oxide particles coated on the surface by reaction with at least one reagent of formula (I),

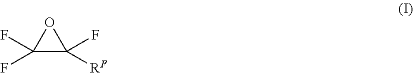

wherein R^{F} is a perfluoro-(C₁ to C₄) alkyl group, particularly trifluoromethyl,
(b) metal oxide particles coated on the surface by reaction with at least one reagent of formula (II),

(R¹O)ₙ(CH₃)₃₋ₙSi—R² (II)

wherein

R¹ is methyl or ethyl,

R² is a (C₁ to C₁₄) alkyl group, particularly a (C₆ to C₁₂) alkyl group, and

n is 1, 2 or 3,

and whose core comprises a liquid, aqueous phase, wherein the powdery composition contains
i) 50 to 90 wt. % of water,
ii) 0.5 wt. % to 10 wt. % of glycerol, and
iii) carboxymethyl starch.

(BB): A powdery composition for temporarily shaping human hair comprising core-shell particles whose shell contains
(a) metal oxide particles coated on the surface by reaction with at least one reagent of formula (Ia),

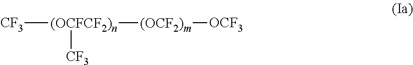

wherein the sum of n+m is a whole number from 20 to 80,
(b) metal oxide particles coated on the surface by reaction with at least one reagent of formula (II),

(R¹O)ₙ(CH₃)₃₋ₙSi—R² (II)

wherein

R¹ is methyl or ethyl,

R² is a (C₁ to C₁₄) alkyl group, particularly a (C₆ to C₁₂) alkyl group, and

n is 1, 2 or 3,

and whose core comprises a liquid, aqueous phase, wherein the powdery composition contains
i) 50 to 90 wt. % of water,
ii) 0.5 wt. % to 10 wt. % of at least one further liquid having a boiling point higher than 150° C. at a pressure of 1013 mbar, chosen from at least one (C₃ to C₈) alcohol having at least two hydroxyl groups, and
iii) carboxymethyl starch.

(CC): A powdery composition for temporarily shaping human hair comprising core-shell particles whose shell contains
(a) metal oxide particles coated on the surface by reaction with at least one reagent of formula (Ia),

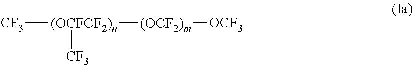

wherein the sum of n+m is a whole number from 20 to 80,
(b) metal oxide particles coated on the surface by reaction with at least one reagent of formula (II),

(R¹O)ₙ(CH₃)₃₋ₙSi—R² (II)

wherein

R¹ is methyl or ethyl,

R² is a (C₁ to C₁₄) alkyl group, particularly a (C₆ to C₁₂) alkyl group, and

n is 1, 2 or 3,

and whose core comprises a liquid, aqueous phase, wherein the powdery composition contains
i) 50 to 90 wt. % of water,
ii) 0.5 wt. % to 10 wt. % of at least one further liquid having a boiling point higher than 150° C. at a pressure of 1013 mbar, chosen from at least one compound from glycerol, 1,3-butylene glycol, 1,4-butylene glycol, 1,2-pentanediol, 2-methyl-2,4-dihydroxypentane and 2-ethyl-1,3-hexanediol, and
iii) carboxymethyl starch.

(DD): A powdery composition for temporarily shaping human hair comprising core-shell particles whose shell contains
(a) metal oxide particles coated on the surface by reaction with at least one reagent of formula (Ia),

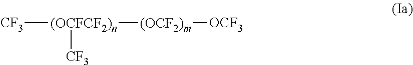

wherein the sum of n+m is a whole number from 20 to 80,
(b) metal oxide particles coated on the surface by reaction with at least one reagent of formula (II),

(R¹O)ₙ(CH₃)₃₋ₙSi—R² (II)

wherein

R¹ is methyl or ethyl,

R² is a (C₁ to C₁₄) alkyl group, particularly a (C₆ to C₁₂) alkyl group, and

n is 1, 2 or 3,

and whose core comprises a liquid, aqueous phase, wherein the powdery composition contains

i) 50 to 90 wt. % of water,

ii) 0.5 wt. % to 10 wt. % of glycerol, and

iii) carboxymethyl starch.

Preferably, each embodiment (A) to (Z) and (AA) to (DD) additionally contains sorbitol.

Preferably, each embodiment (A) to (Z) and (AA) to (DD) additionally contains at least one algae extract and sorbitol.

Preferably, each embodiment (A) to (Z) and (AA) to (DD) additionally contains at least one alkalizing agent, particularly triethanolamine.

Preferably, each embodiment (A) to (Z) and (AA) to (DD) additionally contains at least one algae extract, sorbitol and at least one alkalizing agent, particularly triethanolamine.

The powdery composition according to the invention can also contain auxiliary substances and additives typically added to customary styling aids.

These include cationized protein hydrolysates, wherein the underlying protein hydrolysate can derive from animal sources (e.g., from collagen, milk or keratin), from plant sources (e.g., from wheat, maize, rice, potatoes, soy or almonds), from marine life forms (e.g., from fish collagen or algae), or from protein hydrolysates obtained by biotechnology. Protein hydrolysates underlying cationic derivatives according to the invention can be obtained from the corresponding proteins by chemical, particularly alkali or acid hydrolysis, enzymatic hydrolysis, and/or a combination of both types of hydrolysis. Protein hydrolysis generally gives rise to a protein hydrolysate having a molecular weight distribution from approximately 100 Daltons up to several thousand Daltons. Cationic protein hydrolysates are preferred whose underlying protein component has a molecular weight of 100 to 25,000 Daltons, preferably 250 to 5000 Daltons. Cationic protein hydrolysates also include quaternized amino acids and mixtures thereof. Quaternization of protein hydrolysates or amino acids is frequently performed using quaternary ammonium salts such as N,N-dimethyl-N-(n-alkyl)-N-(2-hydroxy-3-chloro-n-propyl) ammonium halides. Cationic protein hydrolysates can also be derivatized. Plant-based cationic protein hydrolysates and derivatives are preferred.

At least one vitamin, provitamin, vitamin precursor and/or derivative thereof can also be used as a care substance.

Preferred vitamins, provitamins and vitamin precursors according to the invention are conventionally assigned to groups A, B, C, E, F and H. Vitamins belonging to the B group or to vitamin B complex are particularly preferred, most particularly preferably vitamin B₅ (pantothenic acid, panthenol and pantolactone).

At least one plant extract can also be used as a care substance.

These extracts are typically produced by extraction of the entire plant. However, it can also be preferable in individual cases to produce the extracts exclusively from flowers and/or leaves of the plant.

Regarding the preferred plant extracts according to the invention, reference is made in particular to the extracts listed in the table beginning on page 44 of the 3^{rd} edition of the Leitfaden zur Inhaltsstoffdeklaration kosmetischer Mittel (“Guide to the Cosmetic Industry Declaration”), published by the Industrieverband Körperpflege- and Waschmittel e.V. (IKW) (Association for the Personal Care and Detergents Industry), Frankfurt.

Extracts from water lily, green tea, oak bark, stinging nettle, witch hazel, hops, henna, chamomile, burdock, horsetail, whitethorn, lime blossom, almond, aloe vera, pine, horse chestnut, sandalwood, juniper, coconut, mango, apricot, lemon, wheat, kiwi, melon, orange, grapefruit, sage, rosemary, birch, mallow, lady's smock, wild thyme, yarrow, thyme, melissa, restharrow, coltsfoot, marshmallow, meristem, ginseng and ginger root are preferred.

Other suitable care substances are protein hydrolysates and/or derivatives thereof, with protein hydrolysates of vegetable origin (e.g., soy, almond, pea, potato and wheat protein hydrolysates) being preferred. Such products are available, for example, under the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda) and Crotein® (Croda).

Although use of protein hydrolysates as such is preferred, other amino acid mixtures obtained by other means can optionally be used in their place. Use of derivatives of protein hydrolysates, for example, in the form of their fatty acid condensation products, is likewise possible. Such products are sold, for example, under the names Lamepon® (Cognis), Lexein® (Inolex), Crolastin® (Croda), Crosilk® (Croda) or Crotein® (Croda).

The teaching according to the invention naturally includes all isomeric forms, such as cis-trans isomers, diastereomers and chiral isomers.

It is also possible to use a mixture of several protein hydrolysates.

In addition to care substances, further auxiliary substances and additives can also be added.

With addition of a UV filter, the preparations as well as the treated fibers can be protected from damaging influences of UV radiation. It can therefore be advantageous to add at least one UV filter to the powdery compositions. There are no general restrictions on suitable UV filters in terms of their structure and physical properties. In fact, all useful UV filters in the cosmetics sector whose absorption maximum is in the UVA (315-400 nm), UVB (280-315 nm) or UVC (<280 nm) range are suitable. UV filters having an absorption maximum in the UVB range, particularly in the range from approximately 280 to approximately 300 nm, are particularly preferred.

Preferred UV filters can be chosen from substituted benzophenones, p-aminobenzoic acid esters, diphenyl acrylic acid esters, cinnamic acid esters, salicylic acid esters, benzimidazoles and o-aminobenzoic acid esters. 2-Hydroxy-4-methoxybenzophenone-5-sulfonic acid and the sodium salt thereof (Benzophenone-4; Uvinul®MS 40; Uvasorb®S 5) are cited here as examples.

In a particular embodiment the powdery composition further contains one or more substantive dyes. This allows the treated keratinic fibers not only to be temporarily shaped but also be colored at the same time with use of the agent. This can be desirable in particular if only temporary coloration is needed, for example, with striking fashion colors, which can be subsequently removed from the keratinic fibers simply by washing.

Powdery compositions used according to the invention for temporarily shaping keratinic fibers can be produced by simple means. With use of different metal oxide particles coated according to the invention, it has proven effective to first mix all metal oxide particles coated according to the invention. Then, in a separate mixer, all ingredients apart from the metal oxide particles are incorporated into an aqueous phase while stirring. Finally, the coated metal oxide particles are added to the aqueous phase with intensive stirring. The mixing time required is dependent on the mixing energy applied and on the composition of the mixture, but can be from 15 seconds to 5 minutes. If the mixing time is too short, a stable powder is not formed and an oily phase develops. If the mixing time is too long, the powder that initially forms is converted into a pulpy or creamy consistency, and this process is not reversible. It is therefore advisable to determine the optimum mixing time for each system by means of preliminary tests.

The powdery compositions can be packed in almost any container. It must merely be ensured that the mechanical loading of the powder on removal of the composition is not so high that the powder is converted to liquid form as it is removed. Pots, bottles or tetrapacks are suitable, for example, wherein the container can be provided for example with a pouring or metering device. With the use of the powdery composition for temporarily shaping the hair, the desired amount of the powdery composition is first removed from the container. The composition can be applied directly to the hair to be treated or poured into the hand, for example. In the first case the powder applied directly to the hair can be subjected to mechanical loading, with the hands for example, causing the aqueous phase to be released directly onto the hair fibers. If the powdery composition is poured into the hand first, it can first be carefully distributed in the hair and only then subjected to greater mechanical loading, for example by systematically massaging the powder into the hair. An excellent styling effect can be achieved very systematically in this way. It is of course also possible to rub the powdery composition in the hands first and to apply only the resulting liquid or paste-like agent to the hair. This procedure is not preferred, however, since this loses a substantial advantage of the powdery consistency of the styling agent, namely its good distributability. The powdery composition can naturally also be applied with an aid, for instance an applicator, a sponge, a cloth, a brush, or a comb.

A second subject-matter is the use of a powdery composition of the first subject-matter of the invention to give shine to human hair.

A third subject-matter is the use of a powdery composition of the first subject-matter of the invention to temporarily shape human hair.

A fourth subject matter is a method for temporarily shaping and/or giving shine to human hair, wherein before or during application onto human hair, a powdery composition of the first subject-matter of the invention is subjected to mechanical loading, particularly by pressure or friction, releasing a liquid composition, the liquid composition arising from the mechanical loading of the powdery composition action on the hair.

Preferably, the hair is not rinsed after application.

The examples below are intended to illustrate the subject-matter of the present invention without in any way limiting it.

### EXAMPLES

The following formulation was provided as a shine-imparting hair styling powder by mixing. First, the powdery raw materials PW F-MS®, Covapearl Bright 933 AS® and Mica 8 AS R0433® were mixed together. The remaining ingredients were mixed together in a separate container. The powdery raw material mixture was added to this liquid mixture while stirring (propeller stirrer, 650 rpm). Stirring was continued (approx. 10 minutes) until a free-flowing powdery composition formed.

| INGREDIENT | wt. % |
|---|---|
| PWF-MS ® ¹ | 3.00 |
| Covapearl Bright 933 AS ® ² | 2.00 |
| Mica 8 AS R0433 ® ³ | 9.50 |
| Covagel ® ⁴ | 2.00 |
| Fucosorb ® ⁵ | 2.00 |
| Glycerol | 5.00 |
| Triethanolamine | 0.07 |
| Neolone PE ® ⁶ | 0.30 |
| Methylparaben | 0.10 |
| Water | 76.03 |

¹ Nanofine titanium dioxide coated with polyperfluoromethylisopropyl ether (CAS no.: 69991-67-9, EINECS 274-225-4) and triethoxycaprylylsilane (INCI name: Cl 77891, Polyperfluoromethylisopropyl ether, Triethoxycaprylylsilane) (Sensient/LCW)
² Pearlescent mineral pigments coated with triethoxycaprylylsilane, comprising 25-35% titanium dioxide, 65-75% mica, 2% triethoxycaprylylsilane (INCI name: Cl 77891, Mica, Triethoxycaprylylsilane) (Sensient/LCW)
³ Mica coated with triethoxycaprylylsilane, comprising 99% mica, 1% triethoxycaprylylsilane (INCI name: Mica, Triethoxycaprylylsilane) (Sensient/LCW)
⁴ Carboxymethyl starch, sodium salt, with potato starch as the starch source (INCI name: Sodium Carboxymethyl Starch) (Sensient/LCW)
⁵ Algae extract obtained from red and brown algae and combined with sorbitol, containing biopolymeric electrolytes and polysaccharides (INCI name: Algae, Sorbitol) (Sensient/LCW)
⁶ Mixture of 2-phenoxyethanol and methyl isothiazolinone (containing 81-86 wt. % 2-phenoxyethanol; INCI name: Phenoxyethanol, Methylisothiazolinone) (Rohm & Haas)

The hair styling powder obtained was transferred to a polyethylene container. From the container the powder was locally applied to sections of hair of a test subject. The powder adhered to the hair. The sections of hair to which the powder had been applied were massaged with the hand and the aqueous agent in the core of the powder particles was selectively released onto the hair sections. The hair was worked into the desired shape as it was massaged. A hairstyle was obtained whose shape was stabilized by the hair styling powder. The hair appeared shiny.

We claim:

1. Powdery composition for temporarily shaping human hair comprising:
i) 50 to 90 wt. % of water,
ii) 0.5 wt. % to 10 wt. % of at least one further liquid having a boiling point higher than 150° C. at a pressure of 1013 mbar,
iii) at least one starch substituted with carboxy-(C₁ to C₄) alkyl groups, and
iv) metal oxide particles coated on the surface with fluorine-containing organic groups.

2. Powdery composition according to claim 1, wherein the at least one further liquid having a boiling point higher than 150° C. at a pressure of 1013 mbar is chosen from at least one (C₃ to C₈) alcohol having at least two hydroxyl groups.

3. Powdery composition according to claim 1, wherein the at least one further liquid having a boiling point higher than 150° C. at a pressure of 1013 mbar is at least glycerol.

4. Powdery composition according to claim 1, wherein the starch substituted with carboxy-(C₁ to C₄) alkyl groups is chosen from carboxymethyl starch, carboxyethyl starch, carboxypropyl starch and combinations thereof.

5. Powdery composition according to claim 1, wherein the metal oxide particles have been coated on the surface by reaction with at least one reagent of formula (I) 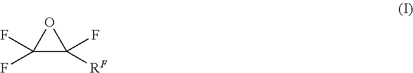
wherein R^{F} is a perfluoro-(C₁ to C₄) alkyl group.

6. Powdery composition according to claim 1, wherein the metal oxide particles are coated on the surface with at least one compound of formula (Ia) 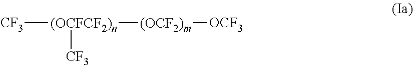
wherein the sum of n+m is a whole number from 20 to 80.

7. Powdery composition according to claim 1 further comprising metal oxide particles coated on the surface by reaction with at least one reagent of formula (II)
(R¹O)ₙ(CH₃)₃₋ₙSi—R² (II)
wherein
R¹ is methyl or ethyl,
R² is a (C₁ to C₁₄) alkyl group, and
n is 1, 2 or 3.

8. Powdery composition according to claim 7, wherein the metal oxide particles have been further coated on the surface by reaction with at least one reagent of formula (Ia) 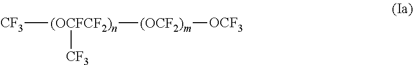
wherein the sum of n+m is a whole number from 20 to 80.

9. Powdery composition according to claim 1, wherein the coated metal oxide particles have a particle diameter of less than 5 μm.

10. Powdery composition according to claim 1, wherein the metal oxide particles coated with fluorine-containing organic groups are chosen from aluminates coated with fluorine-containing organic groups, silicates coated with fluorine-containing organic groups, aluminum silicates coated with fluorine-containing organic groups, titanium dioxide coated with fluorine-containing organic groups, and silica gel coated with fluorine-containing organic groups.

11. Powdery composition according to claim 1, wherein the coated metal oxide particles are present in an amount of 5 wt. % to 25 wt. %, based on total weight of the composition.

12. Powdery composition according to claim 1 further comprising at least one film-forming and/or fixing polymer.

13. Powdery composition according to claim 1 further comprising at least one monosaccharide and/or at least one disaccharide.

14. Powdery composition according to claim 1 further comprising at least one alkalizing agent.

15. Powdery composition according to claim 1 comprising core-shell particles whose shell comprises the metal oxide particles and whose core comprises a liquid, aqueous phase.

16. A method for temporarily shaping and/or giving shine to human hair comprising:
subjecting a powdery composition according to claim 1 to mechanical loading, wherein the mechanical loading occurs before or during application of the powdery composition onto the human hair, thereby releasing a liquid composition from the powdery composition due to the mechanical loading of the powdery composition.

17. Method according to claim 16, wherein the hair is not rinsed following the application.
